SEQUENCE LISTING (109 sequences)

SEQ ID NO: 1 - RNA, 20 nt, synthetic construct
  source 1..20: mol_type = other RNA
tcgtgtatctgagcatgggg

SEQ ID NO: 2 - RNA, 20 nt, synthetic construct
  source 1..20: mol_type = other RNA
gccattcgtgtatctgagca

SEQ ID NO: 3 - RNA, 20 nt, synthetic construct
  source 1..20: mol_type = other RNA
tccaccccatgctcagatac

SEQ ID NO: 4 - RNA, 20 nt, synthetic construct
  source 1..20: mol_type = other RNA
gagcatggggtggaatgacc

SEQ ID NO: 5 - RNA, 20 nt, synthetic construct
  source 1..20: mol_type = other RNA
tgagcatggggtggaatgac

SEQ ID NO: 6 - RNA, 20 nt, synthetic construct
  source 1..20: mol_type = other RNA
tcgtgtatctgagcatgggg

SEQ ID NO: 7 - RNA, 20 nt, synthetic construct
  source 1..20: mol_type = other RNA
cattcgtgtatctgagcatg

SEQ ID NO: 8 - RNA, 20 nt, synthetic construct
  source 1..20: mol_type = other RNA
ccattcgtgtatctgagcat

SEQ ID NO: 9 - RNA, 20 nt, synthetic construct
  source 1..20: mol_type = other RNA
gccattcgtgtatctgagca

SEQ ID NO: 10 - RNA, 20 nt, synthetic construct
  source 1..20: mol_type = other RNA
cccatgctcagatacacgaa

SEQ ID NO: 11 - RNA, 20 nt, synthetic construct
  source 1..20: mol_type = other RNA
cccggtcattccaccccatg

SEQ ID NO: 12 - RNA, 20 nt, synthetic construct
  source 1..20: mol_type = other RNA
tttttctaactggggccatc

SEQ ID NO: 13 - RNA, 20 nt, synthetic construct
  source 1..20: mol_type = other RNA
ccgatacagacaggtatact

SEQ ID NO: 14 - RNA, 20 nt, synthetic construct
  source 1..20: mol_type = other RNA
aacttcggattttttctaac

SEQ ID NO: 15 - RNA, 20 nt, synthetic construct
  source 1..20: mol_type = other RNA
acttcggattttttctaact

SEQ ID NO: 16 - RNA, 20 nt, synthetic construct
  source 1..20: mol_type = other RNA
cttcggattttttctaactg

SEQ ID NO: 17 - RNA, 20 nt, synthetic construct
  source 1..20: mol_type = other RNA
acagacaggtatactcagga

SEQ ID NO: 18 - RNA, 20 nt, synthetic construct
  source 1..20: mol_type = other RNA
ctgagtatacctgtctgtat

SEQ ID NO: 19 - RNA, 20 nt, synthetic construct
  source 1..20: mol_type = other RNA
ttctaactggggccatcctg

SEQ ID NO: 20 - RNA, 20 nt, synthetic construct
  source 1..20: mol_type = other RNA
tctaactggggccatcctga

SEQ ID NO: 21 - RNA, 20 nt, synthetic construct
  source 1..20: mol_type = other RNA
ctaactggggccatcctgag

SEQ ID NO: 22 - RNA, 20 nt, synthetic construct
  source 1..20: mol_type = other RNA
taactggggccatcctgagt

SEQ ID NO: 23 - DNA, 21 bp, synthetic construct
  source 1..21: mol_type = other DNA
gcacaatagcctcggatgtcg

SEQ ID NO: 24 - DNA, 20 bp, synthetic construct
  source 1..20: mol_type = other DNA
ccgttggtatatctgagcat

SEQ ID NO: 25 - RNA, 20 nt, synthetic construct
  source 1..20: mol_type = other RNA
gccctgcatctctgccacat

SEQ ID NO: 26 - RNA, 20 nt, synthetic construct
  source 1..20: mol_type = other RNA
gagcaagatcctcacccatg

SEQ ID NO: 27 - RNA, 20 nt, synthetic construct
  source 1..20: mol_type = other RNA
aggtgtgattggagtgacag

SEQ ID NO: 28 - RNA, 20 nt, synthetic construct
  source 1..20: mol_type = other RNA
gtggaatctcagttcccggc

SEQ ID NO: 29 - RNA, 20 nt, synthetic construct
  source 1..20: mol_type = other RNA
ctgcagaaagaaggtgtgat

SEQ ID NO: 30 - RNA, 20 nt, synthetic construct
  source 1..20: mol_type = other RNA
tgcatctctgccacatgggt

SEQ ID NO: 31 - RNA, 20 nt, synthetic construct
  source 1..20: mol_type = other RNA
cgcagggaaactgctgctcg

SEQ ID NO: 32 - RNA, 20 nt, synthetic construct
  source 1..20: mol_type = other RNA
ccactctgacttcagctgct

SEQ ID NO: 33 - RNA, 20 nt, synthetic construct
  source 1..20: mol_type = other RNA
tgcagcgtcaggctcaggga

SEQ ID NO: 34 - RNA, 20 nt, synthetic construct
  source 1..20: mol_type = other RNA
cccatgctgaggtacaccaa

SEQ ID NO: 35 - RNA, 20 nt, synthetic construct
  source 1..20: mol_type = other RNA
gcagctgtagtcttcttcgg

SEQ ID NO: 36 - RNA, 20 nt, synthetic construct
  source 1..20: mol_type = other RNA
cattggtgtacctcagcatg

SEQ ID NO: 37 - RNA, 20 nt, synthetic construct
  source 1..20: mol_type = other RNA
gaccgggcaaagttgttggc

SEQ ID NO: 38 - RNA, 20 nt, synthetic construct
  source 1..20: mol_type = other RNA
cccattggtgtacctcagca

SEQ ID NO: 39 - RNA, 20 nt, synthetic construct
  source 1..20: mol_type = other RNA
gcagcaggaggagcagctgc

SEQ ID NO: 40 - RNA, 20 nt, synthetic construct
  source 1..20: mol_type = other RNA
cccggtccttttaccccatg

SEQ ID NO: 41 - RNA, 20 nt, synthetic construct
  source 1..20: mol_type = other RNA
accccatgctgaggtacacc

SEQ ID NO: 42 - RNA, 20 nt, synthetic construct
  source 1..20: mol_type = other RNA
ccccatgctgaggtacacca

SEQ ID NO: 43 - RNA, 25 nt, synthetic construct
  source 1..25: mol_type = other RNA
ccatcctgagtatacctgtctgtat

SEQ ID NO: 44 - RNA, 21 nt, synthetic construct
  source 1..21: mol_type = other RNA
ttcatggacgagggcatatac

SEQ ID NO: 45 - RNA, 21 nt, synthetic construct
  source 1..21: mol_type = other RNA
gctgttctgaaacttacgaaa

SEQ ID NO: 46 - RNA, 21 nt, synthetic construct
  source 1..21: mol_type = other RNA
gctgttctgaaacttacgaaa

SEQ ID NO: 47 - RNA, 21 nt, synthetic construct
  source 1..21: mol_type = other RNA
cagatacactgcatcatcttt

SEQ ID NO: 48 - RNA, 21 nt, synthetic construct
  source 1..21: mol_type = other RNA
ccagatacactgcatcatctt

SEQ ID NO: 49 - RNA, 21 nt, synthetic construct
  source 1..21: mol_type = other RNA
gcagaggttctcatcacagtt

SEQ ID NO: 50 - RNA, 21 nt, synthetic construct
  source 1..21: mol_type = other RNA
gagcacaaattggaccctgta

SEQ ID NO: 51 - RNA, 21 nt, synthetic construct
  source 1..21: mol_type = other RNA
gaactagaacagaccaagcaa

SEQ ID NO: 52 - RNA, 21 nt, synthetic construct
  source 1..21: mol_type = other RNA
gaactagaacagaccaagcaa

SEQ ID NO: 53 - RNA, 21 nt, synthetic construct
  source 1..21: mol_type = other RNA
gctttccaaatagtggaactt

SEQ ID NO: 54 - RNA, 21 nt, synthetic construct
  source 1..21: mol_type = other RNA
gctttccaaatagtggaactt

SEQ ID NO: 55 - RNA, 21 nt, synthetic construct
  source 1..21: mol_type = other RNA
cctcccaaagtactgggatta

SEQ ID NO: 56 - RNA, 21 nt, synthetic construct
  source 1..21: mol_type = other RNA
cgagcctattcagacgtttca

SEQ ID NO: 57 - RNA, 21 nt, synthetic construct
  source 1..21: mol_type = other RNA
gtgtatatatgtagcatatca

SEQ ID NO: 58 - RNA, 21 nt, synthetic construct
  source 1..21: mol_type = other RNA
gaggaaatccagcaacatgaa

SEQ ID NO: 59 - RNA, 21 nt, synthetic construct
  source 1..21: mol_type = other RNA
gcctattcagacgtttcactt

SEQ ID NO: 60 - RNA, 21 nt, synthetic construct
  source 1..21: mol_type = other RNA
ctgcagaaagaaggtgtgatt

SEQ ID NO: 61 - RNA, 21 nt, synthetic construct
  source 1..21: mol_type = other RNA
gcaagactcagaagacctctt

SEQ ID NO: 62 - RNA, 21 nt, synthetic construct
  source 1..21: mol_type = other RNA
gaagtcagagtggcaaagcat

SEQ ID NO: 63 - RNA, 21 nt, synthetic construct
  source 1..21: mol_type = other RNA
gctctgacttctaggagcaca

SEQ ID NO: 64 - RNA, 21 nt, synthetic construct
  source 1..21: mol_type = other RNA
tgcttagctccagagcaggat

SEQ ID NO: 65 - RNA, 21 nt, synthetic construct
  source 1..21: mol_type = other RNA
gcgtagtgataagactgattt

SEQ ID NO: 66 - RNA, 21 nt, synthetic construct
  source 1..21: mol_type = other RNA
cagaagtgctggtagcataag

SEQ ID NO: 67 - RNA, 21 nt, synthetic construct
  source 1..21: mol_type = other RNA
gcgtgtggaaagataccataa

SEQ ID NO: 68 - RNA, 21 nt, synthetic construct
  source 1..21: mol_type = other RNA
ggccaaacctgggcaaattta

SEQ ID NO: 69 - RNA, 21 nt, synthetic construct
  source 1..21: mol_type = other RNA
gcgttcttgggaaaccttaaa

SEQ ID NO: 70 - RNA, 21 nt, synthetic construct
  source 1..21: mol_type = other RNA
agccgtgcagcagcttgataa

SEQ ID NO: 71 - RNA, 21 nt, synthetic construct
  source 1..21: mol_type = other RNA
caatcaggcagcatcacattt

SEQ ID NO: 72 - RNA, 21 nt, synthetic construct
  source 1..21: mol_type = other RNA
gaaccagtccgtgctacaaat

SEQ ID NO: 73 - RNA, 21 nt, synthetic construct
  source 1..21: mol_type = other RNA
ggcgaatgcagtagcagtata

SEQ ID NO: 74 - RNA, 21 nt, synthetic construct
  source 1..21: mol_type = other RNA
gcaatcccaaactaccaacaa

SEQ ID NO: 75 - RNA, 21 nt, synthetic construct
  source 1..21: mol_type = other RNA
gcagtttaactcagacttgaa

SEQ ID NO: 76 - RNA, 21 nt, synthetic construct
  source 1..21: mol_type = other RNA
gctgtggtacaatatgaacaa

SEQ ID NO: 77 - RNA, 21 nt, synthetic construct
  source 1..21: mol_type = other RNA
gattccctggagaaactacat

SEQ ID NO: 78 - RNA, 21 nt, synthetic construct
  source 1..21: mol_type = other RNA
attgaacgagaggcaattatt

SEQ ID NO: 79 - RNA, 21 nt, synthetic construct
  source 1..21: mol_type = other RNA
catcgacgatctccatatttc

SEQ ID NO: 80 - RNA, 21 nt, synthetic construct
  source 1..21: mol_type = other RNA
gatggaaacaatcaatcatat

SEQ ID NO: 81 - RNA, 21 nt, synthetic construct
  source 1..21: mol_type = other RNA
cggacattagtattgaagaaa

SEQ ID NO: 82 - RNA, 21 nt, synthetic construct
  source 1..21: mol_type = other RNA
gcagtgaaatacctcttgaat

SEQ ID NO: 83 - RNA, 21 nt, synthetic construct
  source 1..21: mol_type = other RNA
cctcagttatatccgacctat

SEQ ID NO: 84 - RNA, 21 nt, synthetic construct
  source 1..21: mol_type = other RNA
cctcagttatatccgacctat

SEQ ID NO: 85 - RNA, 21 nt, synthetic construct
  source 1..21: mol_type = other RNA
gccacctatgagtctgtcaat

SEQ ID NO: 86 - RNA, 21 nt, synthetic construct
  source 1..21: mol_type = other RNA
gcgctatgaaagaacggagtt

SEQ ID NO: 87 - RNA, 21 nt, synthetic construct
  source 1..21: mol_type = other RNA
gcgctatgaaagaacggagtt

SEQ ID NO: 88 - RNA, 21 nt, synthetic construct
  source 1..21: mol_type = other RNA
ggcaggagaattgcttgaatc

SEQ ID NO: 89 - DNA, 30 bp, synthetic construct
  source 1..30: mol_type = other DNA
aaaaaggatccaccatgggcatggaacatg

SEQ ID NO: 90 - DNA, 37 bp, synthetic construct
  source 1..37: mol_type = other DNA
aaaaagaattctactggtgggtccgatcctgctgcag

SEQ ID NO: 91 - DNA, 39 bp, synthetic construct
  source 1..39: mol_type = other DNA
aaaaagaattcatgagtaaaggagaagctgtgattaaag

SEQ ID NO: 92 - DNA, 40 bp, synthetic construct
  source 1..40: mol_type = other DNA
aaaaagaattcttatttgtatagttcatccatgccaccgg

SEQ ID NO: 93 - DNA, 1497 bp, synthetic construct
  source 1..1497: mol_type = other DNA
atgaagtgggtaacctttatttcccttctttttctctttagctcggcctattccaggggtgtgtttcgtcgagatgggcccgcttacccttacgatgtaccggattacgcactcgacgattccaagggcatgcatagacctggccctcttcccccgagcccacctccaaaggttgatcacaaggcttcccagtggcctcaggagagtgacatggggcagaaggtagcttctttgagtgcgcagtgccacaaccatgatgagagacttgcagagctgacagtcctgcttcagaaactacagatacgggtagaccaagtggatgacggcagggaagggctgtcactgtgggtcaagaatgtggttggacagcacctgcaggagatgggcaccatagaaccacctgatgctaagactgacttcatgactttccaccatgaccatgaagtgcgtctctccaacttggaagatgttcttagaaaactgacagaaaaatctgaggctatccagaaggagctggaagaaaccaagctgaaagcaggcagcagggatgaagagcagcccctccttgaccgtgtgcagcacctagaactggaactgaacctgttgaagtcacagctgtcagactggcagcatctgaagaccagctgtgagcaggctggggcccgcatccaggagactgtgcagctcatgttctctgaggatcagcagggcggttccctcgagtggctattagagaagctttcttctcggttcgtgagcaaggatgagctgcaggtgctcttacatgaccttgagctgaaactgctgcagaatatcacacaccacatcaccgtgacaggacaggccccgacatccgaggctattgtgtctgccgtgaatcaggcagggatttcaggaatcacagaagcgcaagcacatatcattgtgaacaatgctctgaagctgtactcccaagacaagacggggatggtggactttgctctggagtctggaggtggcagcatcctaagcactcggtgctctgagacctatgagaccaagacggcactgctgagcctgtttggggtcccactgtggtacttctcacagtcacctcgagtggtgatccagcccgacatctacccagggaattgctgggcgttcaaaggttcccaggggtacctggtggtgcggttgtccatgaagatctacccaaccacattcaccatggaacacattccaaagacactatcacccactggtaacatctccagtgcccccaaagactttgcagtctatggactggaaacggagtatcaagaagaggggcagcctctgggacggttcacctatgaccaggaaggagactcactccagatgttccacacactggaaagacctgaccaagccttccagatagtagagctccgggtcctgtccaactggggccaccctgagtacacttgcctctaccggttccgagtccacggagagcccatccagaaagatgagttgtag

SEQ ID NO: 94 - protein, 498 aa, synthetic construct
MKWVTFISLLFLFSSAYSRGVFRRDGPAYPYDVPDYALDDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQKDEL

SEQ ID NO: 95 - protein, 25 aa, synthetic construct
MKWVTFISLLFLFSSAYSRGVFRRD

SEQ ID NO: 96 - protein, 9 aa, synthetic construct
YPYDVPDYA

SEQ ID NO: 97 - protein, 456 aa, synthetic construct
DDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 98 - DNA, 49 bp, synthetic construct
  source 1..49: mol_type = other DNA
tcaacaagatgaagagcaccgactcgagttggtgctctgcatcttgttg

SEQ ID NO: 99 - DNA, 48 bp, synthetic construct
  source 1..48: mol_type = other DNA
aggctattgattcgcacattactcgagtaatgtgcgaatcaatagcct

SEQ ID NO: 100 - DNA, 48 bp, synthetic construct
  source 1..48: mol_type = other DNA
gcactgttgattctttcaattggatccaattgaaagaatcaacagtgc

SEQ ID NO: 101 - DNA, 48 bp, synthetic construct
  source 1..48: mol_type = other DNA
gctgttctgaaacttacgaaactcgagtttcgtaagtttcagaacagc

SEQ ID NO: 102 - DNA, 48 bp, synthetic construct
  source 1..48: mol_type = other DNA
gcctattcagacgtttcacttctcgagaagtgaaacgtctgaataggc

SEQ ID NO: 103 - protein, 728 aa, synthetic construct
MKWVTFISLLFLFSSAYSRGVFRRDGPSGGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKTGGDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 104 - protein, 25 aa, synthetic construct
MKWVTFISLLFLFSSAYSRGVFRRD

SEQ ID NO: 105 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 106 - protein, 453 aa, synthetic construct
DDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 107 - protein, 4 aa, synthetic construct
KDEL

SEQ ID NO: 108 - RNA, 21 nt, synthetic construct
  source 1..21: mol_type = other RNA
ctctcaggatgataacgattt

SEQ ID NO: 109 - RNA, 21 nt, synthetic construct
  source 1..21: mol_type = other RNA
atcgttatcatcctgagagta

(19) United States
(12) Patent Application Publication
(10) Pub. No.: US 2026/0078369 A1
(43) Pub. Date: Mar. 19, 2026

(54) Method of Inhibiting Durotaxis and/or Treating Fibrosis

(71) Applicant: Agency for Science, Technology and Research, Singapore (SG)

(72) Inventors: Yin Loon Lee, Singapore (SG); Brian Edmund Burke, Singapore (SG); Keng Hwee Chiam, Singapore (SG); Myeongjun Jun, Singapore (SG); Colin Stewart, Singapore (SG); Xianrong Wong, Singapore (SG); Srikala Raghavan, Singapore (SG); Avinanda Banerjee, Singapore (SG); Krystle Joy Ng, Singapore (SG)

(21) Appl. No.: 19/108,567

(22) PCT Filed: Sep. 5, 2023
(86) PCT No.: PCT/SG2023/050603

§ 371 Date: Mar. 4, 2025

(30) Foreign Application Priority Data
Sep. 5, 2022 (SG) 10202250904E

(51) Int. Cl. C12N 15/113; A61K 38/17; A61P 1/16; C12N 9/22; C12N 15/11

(52) U.S. Cl. CPC C12N 15/113; A61K 38/1709; A61P 1/16; C12N 9/226; C12N 15/111; C12N 2310/14; C12N 2310/20

(57) ABSTRACT

The present invention relates to cell biology. In particular, the present invention teaches methods of contacting the cells with a LING complex inhibitor to inhibit durotaxis and, in consequence, reduce fibrosis and metastasis. Methods of treating wound healing are also disclosed herein.

### FIELD

The present invention relates to cell biology. In particular, the present invention teaches methods of inhibiting durotaxis and/or treating fibrosis. Methods of treating wound healing are also disclosed herein.

### BACKGROUND

Fibrosis is the abnormal accumulation of fibrous tissue that can occur as a part of the wound-healing process in response to various types of injuries and diseases. Examples of fibrosis include liver fibrosis, lung fibrosis (such as pulmonary fibrosis), skin fibrosis, or kidney fibrosis (including diabetic nephropathy), and can affect all types of tissues and organs. This abnormal tissue remodelling can affect the structure and function of the affected areas, leading to organ dysfunction and various health complications. Fibrosis can also influence cancer metastasis and accelerate chronic graft rejection in transplant recipients. There is currently a lack of effective treatments that directly target the mechanism of fibrosis.

Durotaxis is a type of cell migration where cells migrate from a softer to a stiffer substrate. Durotaxis has been thought to be associated with various pathological processes, including fibrosis, wound healing, tissue regeneration and cancer metastasis. However, molecular regulators of durotaxis are not well characterized or understood. There is also no known or effective inhibitors that can target durotaxis.

TGF-β is a cytokine that is known to play an important role in regulating fibrosis. It is secreted by various cell types, including immune cells, and acts on a wide range of cell types, including fibroblasts. TGF-β also stimulates the production of various fibrogenic factors and cytokines, which contribute to the fibrotic process. It is therefore a target for the therapeutic intervention of fibrosis.

It would be desirable to overcome or ameliorate at least one of the above-described problems, or at least to provide a useful alternative.

### SUMMARY

Disclosed herein is a method of inhibiting durotaxis of a cell, the method comprising contacting the cell with a Linker of Nucleoskeleton and Cytoskeleton (LINC) complex inhibitor.

Disclosed herein is a method of preventing or treating a disease associated with fibrosis in a subject, the method comprising administering an effective amount of a LINC complex inhibitor to the subject.

Disclosed herein is a method of inhibiting or preventing metastasis of a cancer in a subject, the method comprising administering an effective amount of a LINC complex inhibitor to the subject.

Disclosed herein is a method of treating a metastatic cancer in a subject, the method comprising administering an effective amount of a LINC complex inhibitor to the subject.

Disclosed herein is a method of treating or preventing a disease associated with inflammation in a subject, the method comprising administering an effective amount of a LINC complex inhibitor to the subject.

Disclosed herein is a method of promoting wound healing or tissue regeneration in a subject, the method comprises administering an effective amount of a LINC complex inhibitor to the subject.

Disclosed herein is a method of treating a wound in a subject, the method comprises administering an effective amount of a LINC complex inhibitor to the subject.

### BRIEF DESCRIPTION OF DRAWINGS

Embodiments of the present invention are hereafter described, by way of non-limiting example only, with reference to the accompanying drawings in which:

FIG. 1. A stepwise fabrication method for making stepped stiffness gradient pillar arrays without modifying dimensions of the structure. (a) Fabrication method of making stepped stiffness gradient monolayer (b) Left and middle: Confirmation of the stiffness border using bright field and fluorescence imaging. Right: Intensity profile of the scanned area. Scale bar—5 m (c) Top and side view of the stiffness border by scanning electron microscopy (SEM). (d) Confirmation of the stiffness border using atomic force microscopy (AFM).

FIG. 2. Immunofluorescence staining of an immortalized fibroblast cell line expressing HA-tagged dominant negative Sun1 (DNSun1) under control of a doxycycline-inducible promoter following treatment with and without 1 μg/l doxycycline. Cells are co-stained with Hoescht for DNA, rat anti-HA antibody to detect DNSun1, and mouse anti-Nesprin-2 antibody to detect nuclear envelope localization of Nesprin-2. DNA staining with Hoescht marks cell nuclei. In the middle column, in untreated cells, there is negligible expression of DNSun1 while in doxycycline-treated cells, there is overexpression of DNSun1. In the right-hand column, in untreated cells, Nesprin-2 remains localized to the nuclear envelope, while in doxycycline-treated cells, Nesprin-2 is mislocalized from the nuclear envelope to the cytoplasm.

FIG. 3. LINC complex disruption in fibroblasts does not affect migration-related behavior on uniform substrate stiffness but inhibits durotaxis. (a, b) Fluorescence image of fibroblasts expressing dominant-negative Sun1 (DNSun1), showing fluorescent protein-tagged cytoskeletal proteins and deflection of pillars on soft (21 nN/mm) and hard (53 nN/mm) pillars. Left: actin, middle: vinculin, right: magnified image of vinculin and traction force from the insets in the middle panel. c-e, Average values for pillar deflection (c), traction force (d) and focal adhesion (FA) area (e) of wild type and DNSun1 cells on different substrate stiffness. N=15 for each data point. Error bars represent standard deviation. There is no significant difference in these parameters for wildtype and DNSun1 cells. (f) Durotaxis ratio of wildtype and DNSun1 cells around the 21 nN/mm-53 nN/mm interface area shows presence of durotaxis in wildtype and cells harbouring but not expressing the DNSun1 transgene. Induction of DNSun1 expression results in no durotaxis (durotactic ratio=0.5, implying random migration) in cells expressing DNSun1. (Number of experiments, Nₑₓₚ=3 for wildtype and Nₑₓₚ=5 DNSun1 cells). (g) Representative time-lapse images of a DNSun1-expressing cell around the stiffness border of a membrane with 21 nN/m (soft) and 53 nN/m (stiff) substrates. Arrows in insets (i-iv) show traction forces exerted on the pillars for area of the cell at the interface between stiffnesses, or the leading or trailing edge of the cell as it migrates towards the softer substrate. (h) Line plots of traction forces at the interface and leading-trailing edge when a DNSun1 cell migrates toward the soft substrate. (i) Boxplots of traction forces at the interface and leading-trailing edge when a wildtype cell migrates from soft to stiff substrate, or when a DNSun1 cell migrates toward the soft substrate. (j) Line plots of traction forces at the interface and leading-trailing edge when a DNSun1 cell migrates toward the stiff substrate. (k) Boxplots of traction forces at the interface and leading-trailing edge when a wildtype or DNSun1 cell migrates from soft to stiff substrate. Dark grey and light grey arrows (h-k) indicate the direction of migration in wildtype and DNSun1 cells respectively. Expression of DNSun1 results in increased traction forces on the softer substrate when cells are migrating from stiff to soft substrate. In other contexts, DNSun1 does not affect traction forces exerted by the cell. For line plots, n=1 cell. For box plots, n=10 cells for wildtype MEFs and n=5 cells for DNSun1 cells per box plot. Error bars represent standard deviation. P-values were calculated using two-sample t-test (***p<0.001; ns for not significant). Scale bars, 20 m and 10 nN of the traction force, respectively.

FIG. 4. (a) Graph showing magnitude of the difference in traction forces (imbalanced force) for the cell's distal area on the soft (21 nN/m) and stiff (53 nN/m) substrates, comparing wild-type and DNSun1-expressing cells. N=10 cells for wildtype and DNSun1 DN. P-values were calculated using two-sample t-test. ***p<0.001. (b) Histogram of the distance between the nucleus and centroid of the cell around the stiffness border area. Inset: representative image of the wildtype and SUN1 DN cell. Insets: images of cells around the stiffness border. Grey and white lines indicate the trajectory of the centroid of the cell and nucleus respectively. (c) Graph showing magnitude of imbalanced traction forces (grey bar) and durotaxis ratio (black bar) for wildtype and DNSun1-expressing cells experiencing the indicated dual stiffness regimes (21-53, 24-28, 43-53 nN/m).

FIG. 5. Lung tissue were isolated from Sun1^{tm1c/tm1c};Sun2^{-/-};Rosa26^{MerCreMer} (Sun1^{fl/fl}, Sun2KO) mice treated with vehicle (−TMX, corn oil) or tamoxifen (+TMX). Lung tissue was lysed, run on SDS-PAGE and immunoblotted for Sun1 and beta-actin. In tissue from vehicle treated mice, there is robust expression of Sun1, which is considerably reduced in tissue from TMX-treated mice. IgH indicates immunoglobulin heavy chain, detected by anti-mouse secondary antibody used to detect anti-Sun1 mouse monoclonal antibody.

FIG. 6. 6-week-old mice of the indicated genotypes were treated with oil as a vehicle control, or tamoxifen to induce loss of Sun1, so as to obtain mice that were effectively Sun1/Sun2 wildtype, Sun1 “knockout”, Sun2 “knockout” or Sun1/Sun2 “double knockout”. 2 weeks later, the airways of these mice were treated with bleomycin to induce pulmonary fibrosis. They, along with untreated wildtype mice, were sacrificed three weeks after bleomycin treatment to collect lung tissue. (A) Lungs were prepared for histopathological evaluation by Ashcroft scoring. Bleomycin-treated Sun2 knockout and Sun1/Sun2 double knockout mice had reduced lung fibrosis as indicated by Ashcroft score, compared to bleomycin-treated Sun1/Sun2 wildtype mice. (B) Lung tissue were analyzed for hydroxyproline content, a measure for deposition of collagen extracellular matrix, which is a marker for fibrosis. Lungs from bleomycin-treated Sun1/Sun2 double knockout mice had reduced levels of collagen compared to bleomycin-treated Sun1/Sun2 wildtype mice, consistent with reduced fibrosis.

FIG. 7. Male (M) 6-week-old Sun1^{flox/flox}, Rosa26^{MerCreMer/MerCreMer} mice of the indicated Sun2 genotypes (wildtype, WT or knockout, KO) were treated with oil as a vehicle control, or tamoxifen (TMX) to induce loss of Sun1, so as to obtain mice that were effectively Sun1/Sun2 wildtype, Sun1 “knockout”, Sun2 “knockout” or Sun1/Sun2 “double knockout”. 2 weeks later, the airways of these mice were treated with bleomycin to induce pulmonary fibrosis. 3 weeks after bleomycin treatment, BAL (bronchoalveolar lavage) fluid collected from lung via tracheal cannulation were analysed for quantities of immune cells. Normal Control (inhouse data) are data from wildtype mice not treated with bleomycin. Total immune cell count was significantly reduced in bleomycin-treated Sun1/Sun2 double knockout mice compared to Sun1/Sun2 wildtype mice. Neutrophil count was significantly reduced in bleomycin-treated Sun1 knockout, Sun2 knockout and Sun1/Sun2 double knockout mice compared to Sun1/Sun2 wildtype mice, whereas there was little effect of LINC complex disruption on lymphocyte and macrophage count following bleomycin treatment.

FIG. 8. Male (M) 6-week-old Sun1^{flox/flox}, Rosa26^{MerCreMer/MerCreMer} mice of the indicated Sun2 genotypes (wildtype, WT or knockout, KO) were treated with oil as a vehicle control, or tamoxifen to induce loss of Sun1, so as to obtain mice that were effectively Sun1/Sun2 wildtype, Sun1 “knockout”, Sun2 “knockout” or Sun1/Sun2 “double knockout”. 2 weeks later, the airways of these mice were treated with bleomycin to induce pulmonary fibrosis. 3 weeks after bleomycin treatment, BAL (bronchoalveolar lavage) fluid collected from lung via tracheal cannulation were analysed for quantities of the IL-6 cytokine. Normal Control (inhouse data) are data from wildtype mice not treated with bleomycin. IL-6 levels were significantly reduced in bleomycin-treated Sun1 knockout, Sun2 knockout and Sun1/Sun2 double knockout mice compared to Sun1/Sun2 wildtype mice.

FIG. 9. Genetic disruption of the LINC complex ameliorates liver steatosis and fibrosis in a NASH model. Mice with the following genotypes were put on a methionine-choline deficient diet for 8 weeks: Sun1^{tm1c/tm1c};Sun2^{+/+};Rosa26^{MerCreMer}, oil vehicle treated (wildtype, WT), Sun1^{tm1c/tm1c};Sun2^{+/+};Rosa26^{MerCerMer} tamoxifen treated (Sun1 knockout, S1), Sun1^{tm1c/tm1c};Sun2^{-/-};Rosa26^{MerCreMer} oil vehicle treated (Sun2 knockout, S2), Sun1^{tm1c/tm1c};Sun2^{-/-};Rosa26^{MerCreMer} tamoxifen treated (Sun1/2 double knockout, DKO). Control mice were wildtype mice on normal chow (WT-). Mice were sacrificed, livers isolated and subject to histological analysis. (A) Livers were stained with Masson's trichrome. Steatosis was observable as lacunae in WT and S1 groups, while S2 and DKO groups had minimal steatosis and were comparable to WT- group. (B) Livers were stained with Oil Red O. Images were processed to isolate Oil Red stain. As there was no steatosis in WT- group, no Oil Red O staining is visible. Steatosis was observable as red signal in the original samples (greyscale in the image) in WT and S1 groups, while S2 and DKO groups had minimal Oil Red O staining, indicating reduced steatosis. (C) Picrosirius red staining was carried out on liver sections, indicating collagen deposition and fibrosis. (D) Quantification of Picrosirius red staining revealed increase in fibrosis in WT group compared to WT- as expected. No statistically significant difference was observed between WT and S1 groups, whereas fibrosis was significantly reduced in S2 group. Fibrosis was also reduced in DKO group compared to WT group. * indicates p<0.05.

FIG. 10. Sun2 RNAi attenuates TGFb-mediated smooth muscle actin expression. Human fetal lung fibroblasts (IMR-90) and murine hepatic stellate cells were transduced with control shRNA, or shRNA against Sun1 or Sun2, and treated with vehicle control or TGFβ to induce conversion to fibrosis-associated myofibroblasts. Cells were lysed, and lysates run on SDS PAGE followed by transfer and Western blotting for Sun1, Sun2, GAPDH and smooth muscle actin. Top left panel shows loss of Sun1 protein following lentiviral-mediated shRNA (short hairpin RNA) depletion of Sun1 (shSun1), and loss of Sun2 protein following shRNA depletion of Sun2 (shSun2) in IMR-90 human fetal lung fibroblasts. Bottom left panel shows robust induction of smooth muscle actin (SMA) following TGFβ treatment in scrambled control or Sun1 shRNA conditions, but minimal induction of SMA in Sun2 shRNA conditions, in IMR-90 cells. Top right panel shows depletion of Sun1 in shSun1 and depletion of Sun2 in shSun2 in murine hepatic stellate cells. Bottom right panel shows robust induction of SMA following TGFb treatment in scrambled control and shSun1 samples, but no induction of SMA in shSun2 sample.

FIG. 11. (A, B) Human foreskin dermal fibroblasts were transduced with control shRNA, or shRNA against Sun1 or Sun2 (A). Human fetal lung fibroblasts, human foreskin dermal fibroblasts and murine hepatic stellate cells were transduced with lentivirus expressing DNSUN1 under control of a doxycycline-inducible promoter (B). DNSUN1 and untransduced control cells were treated with doxycycline. Control cells or cells transduced with the aforementioned constructs were treated with vehicle control or with TGFβ to induce conversion to fibrosis-associated myofibroblasts. Cells were lysed, and lysates run on SDS PAGE followed by transfer and Western blotting for GAPDH and smooth muscle actin. Loss of Sun2 or overexpression of DNSUN1 attenuated smooth muscle actin expression.

FIG. 12. Following transduction with lentiviruses expressing non-targeting (Off Target), Sun1 (ShSun1) or Sun2 (ShSun2) shRNA, Huh-7 hepatocyte cells were seeded at equal cell numbers to achieve confluent densities and cultured with BODIPY™ FL C₁₆ to promote accumulation. Cells were fixed and imaged. Quantification of BODIPY FL C₁₆ staining indicated a statistically significant reduction in mean intensity per field of view in the ShSun2 group.

FIG. 13. Lipofection of siRNA in vivo leads to depletion of Sun2 protein in the liver (A) Liver tissue dissected from control siRNA or Sun2 siRNA injected C57BL/6NTac mice 5 days and 7 days after the treatment. Tissue lysates were analysed by Western blot and probed with anti-Sun2 and anti-GAPDH as a loading control. A significant reduction in Sun2 protein was detected. (B) Signal intensity was quantified using Fiji software. For each sample, Sun2 signal intensity was divided by GAPDH signal intensity. Statistical significance was determined by comparing Sun2 siRNA injected to control siRNA injected samples using unpaired t test. *P<0.05, **P<0.01.

FIG. 14. Depletion of Sun2 by siRNA reduces liver steatosis in a NASH model. C57B6 mice were fed on normal chow or on a methionine-choline deficient (MCD) diet for 8 weeks. Mice were injected weekly with control (Off Target) or Sun2 siRNA. Mice were then sacrificed and livers isolated for Oil Red O staining. Sun2 depletion by siRNA was observed to reduce Oil Red O levels in the livers of MCD diet-fed mice, compared to control siRNA.

FIG. 15. Genetic disruption of the LINC complex from loss of Sun1 and Sun2 promotes wound healing in the skin. Representative digital images of control and Sun1/2 double knockout (DKO) wounds from Day 0-Day 6.

FIG. 16. Loss of Sun1 and Sun2 protein in Sun1/2 DKO skin. Control and Sun1/2 double knockout (DKO) skin were immunostained for SUN1, SUN2 and counterstained with DAPI for DNA. Nuclear envelope-localized Sun1 and Sun2 was observed in control, but not in Sun1/2 DKO skin.

FIG. 17. Schematic to show how area of wound was measured and how wound contraction was calculated.

FIG. 18. Genetic disruption of the LINC complex from loss of Sun1 and Sun2 promotes wound healing in the skin. Bar graph representing percentage of wound closure in control and Sun double knockout (DKO) animals on Day 6 after wounding. For WT, N=9 mice; n=18 wounds; for DKO, N=10 mice; n=20 wounds (WT). **, p<0.01

FIG. 19. Haematoxylin and Eosin (H&E) staining of D6 wildtype (WT) and SUN double knockout (DKO) wounds. Dashed lines denote the wound margin.

FIG. 20. Masson Trichrome staining of D6 wildtype (WT) and SUN double knockout (DKO) wounds. Dashed lines denote

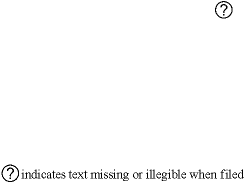

the wound margin.

FIG. 21. Loss of Sun1 reduces primary tumour size in PyMT tumour mouse model. Mean mammary tumour volume per mouse in PyMT Sun1^{+/+} (FVB/N), PyMT Sun1^{+/+}(Mixed C57BL/6/FVB/N) and PyMT Sun1^{-/-} (Mixed C57BL/6/FVB/N) transgenic tumors. Values are averages +/−SEM from n=6 female mice per genotype. The difference between mean mammary tumour volume between PyMT Sun1^{-/-} (Mixed C57BL/6/FVB/N) and PyMT Sun1^{+/+} (Mixed C57BL/6/FVB/N) transgenic tumors was significant (***P<0.05 by unpaired 2-tail t-test).

FIG. 22. Loss of Sun1 reduces metastasis to the lung in PyMT tumour mouse model. RT-PCR quantitation of relative PyMT expression levels in the lung of PyMT Sun1⁺⁺ (FVB/N), PyMT Sun1^{+/+} (Mixed C57BL/6/FVB/N) and PyMT Sun1^{-/-} (Mixed C57BL/6/FVB/N), normalized to the RPLP0 housekeeping gene from n=3 female mice per genotype. Relative PyMT expression in the lung in PyMT Sun1^{-/-} ((Mixed C57BL/6/FVB/N) was significantly reduced compared to PyMT Sun1^{+/+} (Mixed C57BL/6/FVB/N) (***P<0.005 by unpaired 2-tail t-test). Error bars are SEM.

### DETAILED DESCRIPTION

The specification teaches a method of inhibiting durotaxis of a cell. The specification also teaches a method of preventing or treating fibrosis or a disease associated with fibrosis in a subject.

Disclosed herein is a method of inhibiting durotaxis of a cell, the method comprising contacting the cell with a Linker of Nucleoskeleton and Cytoskeleton (LINC) complex inhibitor.

Without being bound by theory, the inventors have shown that LINC complex disruption, such as via viral-mediated overexpression of a dominant negative SUN1 transgene (DNSUN1), blocks durotaxis, a form of directed cell migration from soft to stiff substrates that is correlated with fibrosis. It was further demonstrated in mice that genetic disruption of the LINC complex such as via ablation of either Sun2 or both Sun1 and Sun2 ameliorates lung fibrosis in a bleomycin-induced model of idiopathic pulmonary fibrosis. This presents a general strategy for treating fibrosis and associated diseases. The inventors further show that LINC complex disruption via RNAi depletion of SUN2 inhibits TGFβ signaling, a key driver of fibrosis in lung, skin and liver cells. Furthermore, genetic disruption of the LINC complex via ablation of either SUN2 or both SUN1 and SUN2 in mice ameliorates liver steatosis and liver fibrosis induced by methionine-choline deficient diet, a model for non-alcoholic steatohepatitis (NASH). Finally, evidence is provided that depletion of SUN2 or overexpression of a dominant negative SUN1 transgene (DNSUN1) in lung, skin and liver cells blocks fibrosis mediated by TGFb signaling.

The term “inhibitor” as used herein refers to an agent that decreases or inhibits at least one function or biological activity of a target molecule. For example, a LINC complex inhibitor may decrease or reduce at least one function or biological activity of LINC complex. A LINC complex inhibitor may inhibit the formation of a LINC complex (i.e. inhibition of LINC complex assembly), promote disruption/degradation of a LINC complex, or inhibit LINC complex activity/function.

Aspects of the present invention comprise LINC complex inhibition using a LINC complex inhibitor.

A “LINC complex inhibitor” refers to any agent capable of achieving LINC complex inhibition. LINC complex inhibitors include agents capable of inhibiting formation of a LINC complex (i.e. inhibiting LINC complex assembly), disrupting/degrading a LINC complex, or inhibiting LINC complex function.

In one embodiment, the LINC complex inhibitor is selected from a nucleic acid molecule, a polypeptide and a small molecule. In one embodiment, the nucleic acid molecule is a DNA and/or RNA.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The term “nucleotide” refers to a ribonucleotide or a deoxyribonucleotide or modified form thereof, as well as an analogue thereof. Nucleotides include species that comprise purines, e.g., adenine, hypoxanthine, guanine, and their derivatives and analogues, as well as pyrimidines, e.g., cytosine, uracil, thymine, and their derivatives and analogues.

Nucleotide analogues include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term “nucleotide” is also meant to include what are known in the art as universal bases. By way of example, universal bases include but are not limited to 3-nitropyrrole, 5-nitroindole, or nebularine. The term “nucleotide” is also meant to include the NY to P5′ phosphoramidate, resulting from the substitution of a ribosyl 3′ oxygen with an amine group. Further, the term nucleotide also includes those species that have a detectable label, such as for example a radioactive or fluorescent moiety, or mass label attached to the nucleotide.

As used herein, the terms “polypeptide”, “peptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogues of an amino acid including, for example, unnatural amino acids or polypeptides with substituted linkages.

The terms “disruption” and “disrupted” are used interchangeably herein to refer to any genetic modification that decreases or eliminates expression and/or the functional activity of the nucleic acid or an expression product thereof. For example, disruption of a gene includes within its scope any genetic modification that decreases or eliminates expression of the gene and/or the functional activity of a corresponding gene product (e.g. mRNA and/or protein). Genetic modifications include complete or partial inactivation, suppression, deletion, interruption, blockage, or down-regulation of a nucleic acid (e.g., a gene). Illustrative genetic modifications include, but are not limited to, gene knockout, inactivation, mutation (e.g., insertion, deletion, point, or frameshift mutations that disrupt the expression or activity of the gene product), or use of inhibitory nucleic acids (e.g., inhibitory RNAs such as sense or antisense RNAs, molecules that mediate RNA interference such as siRNA, shRNA, miRNA; etc.), inhibitory polypeptides (e.g., antibodies, polypeptide-binding partners, dominant negative polypeptides, enzymes etc.) or any other molecule that inhibits the activity of the gene or level or functional activity of an expression product of the gene.

In some embodiments, formation of a LINC complex may be inhibited by inhibiting the gene and/or protein expression of a constituent protein of a LINC complex. Constituent proteins of LINC complexes include SUN domain-containing proteins and KASH domain-containing proteins. A constituent protein of a LINC complex may be referred to as “a LINC complex protein”. A LINC complex protein may be SUN1, SUN2, Nesprin-1, Nesprin-2, Nesprin-3, Nesprin-4 or KASH5.

In one embodiment, the LINC complex inhibitor is capable of binding to a LINC complex, a LINC complex protein or an interaction partner for a LINC complex protein, or wherein the LINC complex inhibitor is capable of reducing expression of a LINC complex protein.

In one embodiment, the LINC complex inhibitor is capable of inhibiting interaction between a LINC complex protein and an interaction partner for a LINC complex protein. In one embodiment, the LINC complex inhibitor is capable of inhibiting or disrupting interaction between a SUN domain-containing protein and a KASH domain-containing protein.

In some embodiments, a LINC complex inhibitor inhibits protein-protein interaction between: SUN1 and Nesprin-1, SUN2 and Nesprin-1, SUN1 and Nesprin-2, SUN1 and Nesprin-3, SUN1 and Nesprin-4, SUN1 and KASH5, SUN2 and Nesprin-2, SUN2 and Nesprin-3, SUN2 and Nesprin-4 and SUN2 and KASH5.

The LINC complex inhibitor may be a nucleic acid molecule, a polypeptide or a small molecule.

In one embodiment, the LINC complex inhibitor is a polypeptide derived from a LINC complex protein. In one embodiment, the LINC complex inhibitor is a polypeptide derived from SUN1 and/or SUN2. The polypeptide may disrupt SUN-KASH interactions by competing with endogenous Sun1 and/or Sun2 proteins for binding to their cognate KASH domain proteins. In one embodiment, the LINC complex inhibitor is a polypeptide derived from Nesprin-1, Nesprin-2, Nesprin-3, Nesprin-4 and/or KASH5. The polypeptide may disrupt SUN-KASH interactions by competing with endogenous Nesprin-1, Nesprin-2, Nesprin-3, Nesprin-4 and/or KASH5 proteins for binding to their cognate Sun domain proteins. A polypeptide LINC complex inhibitor may be one that is described in WO2023101607 (which is hereby incorporated by reference in its entirety).

In one embodiment, the LINC complex inhibitor is capable of modifying a gene encoding a LINC complex protein to reduce its expression. The LINC complex inhibitor may comprise a site-specific nuclease (SSN) targeting a gene encoding a LINC complex protein.

In one embodiment, the LINC complex inhibitor is an inhibitory nucleic acid capable of reducing expression of a LINC complex protein by RNA interference (RNAi).

In one embodiment, the LINC complex inhibitor is an inhibitor that is described in WO2019143300, or WO2021010898 (which are hereby incorporated by reference in their entirety).

The LINC complex inhibitor may be an inhibitory nucleic acid molecule or a site-specific nuclease (SSN) system that is capable of disrupting a gene encoding a LINC complex protein. The disruption of the gene encoding the LINC complex protein may lead to a decrease in the expression of the gene. The decrease may be, for example, a decrease of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or up to and including a 100% decrease or any decrease between 5-100% as compared to a reference level.

In some embodiments, a nucleic acid inhibitor according to the present disclosure may be an antisense nucleic acid as described herein. In some embodiments, a nucleic acid inhibitor may comprise an antisense nucleic acid as described herein. In some embodiments, a nucleic acid inhibitor may encode an antisense nucleic acid as described herein.

As used herein, an ‘antisense nucleic acid’ refers to a nucleic acid (e.g. DNA or RNA) that is complementary to at least a portion of a target nucleotide sequence (e.g. of RNA encoding a target gene described herein). Antisense nucleic acids according to the present disclosure are preferably single-stranded nucleic acids, and bind via complementary Watson-Crick base-pairing to a target nucleotide sequence. Complementary base-pairing may involve hydrogen bonding between complementary base pairs. Antisense nucleic acids may be provided as single-stranded molecules, as for example in the case of antisense oligonucleotides, or may be comprised in double-stranded molecular species, as for example in the case of siRNA, shRNA and pre-miRNA molecules.

Complementary base-pairing between the antisense nucleic acid and its target nucleotide sequence may be complete. In such embodiments the antisense nucleic acid comprises, or consists of, the reverse complement of its target nucleotide sequence, and complementary base-pairing occurs between each nucleotide of the target nucleotide sequence and complementary nucleotides in the antisense nucleic acid. Alternatively, complementary base-pairing between the antisense nucleic acid and its target nucleotide sequence may be incomplete/partial. In such embodiments complementary base-pairing occurs between some, but not all, nucleotides of the target nucleotide sequence and complementary nucleotides in the antisense nucleic acid.

Such binding between nucleic acids through complementary base pairing may be referred to as ‘hybridisation’. Through binding to its target nucleotide sequence, an antisense nucleic acid may form a nucleic acid complex comprising (i) the antisense nucleic acid and (ii) a target nucleic acid comprising the target nucleotide sequence.

The nucleotide sequence of an antisense nucleic acid is sufficiently complementary to its target nucleotide sequence such that it binds or hybridises to the target nucleotide sequence. It will be appreciated that an antisense nucleic acid preferably has a high degree of sequence identity to the reverse complement of its target nucleotide sequence. In some embodiments, the antisense nucleic acid comprises or consists of a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of its target nucleotide sequence.

In some embodiments, a nucleic acid inhibitor is an antisense oligonucleotide (ASO). ASOs are single-stranded nucleic acid molecules comprising or consisting of an antisense nucleic acid to a target nucleotide sequence. An antisense oligonucleotide according to the present disclosure may comprise or consist of an antisense nucleic acid as described herein.

ASOs can modify expression of RNA molecules comprising their target nucleotide sequence by altering splicing, or by recruiting RNase H to degrade RNA comprising the target nucleotide sequence. RNase H recognises nucleic acid complex molecules formed when the ASO binds to RNA comprising its target nucleotide sequence. ASOs according to the present disclosure may comprise or consist of an antisense nucleic acid according to the present disclosure. ASOs may comprise 10 to 40 (e.g. 17 to 30, 20 to 27, 21 to 23) nucleotides in length. Many ASOs are designed as chimeras, comprising a mix of bases with different chemistries, or as gapmers, comprising a central DNA portion surrounded by ‘wings’ of modified nucleotides. ASOs sometimes comprise alterations to the sugar-phosphate backbone in order to increase their stability and/or reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′ O-methyl (2′ OMe) and 2′-O-methoxyethyl (MOE) ribose modifications and/or 5′-methylcytosine modifications.

The nucleic acid inhibitor may be an RNAi agent (e.g. siRNA, shRNA or miRNA-based shRNA or gRNA for CRISR/CAS9 knockout) or a nucleic acid encoding an RNAi agent that reduces expression of a gene/mRNA.

The term “RNAi agent” or “RNAi” as used interchangeably herein, refer to an agent that contains RNA as that term is defined herein, and which mediates the targeted cleavage of an RNA transcript via an RNA-induced silencing complex (RISC) pathway. RNAi agent directs the sequence-specific degradation of mRNA through a process known as RNA interference (RNAi). The RNAi agent modulates, e.g., inhibits, the expression of a gene in a cell, e.g., a cell within a subject, such as a mammalian subject. The term “RNAi agent” includes both shRNAs, or precursor RNAs that are processed by RISC into siRNAs, as well as the siRNAs themselves that inhibits the expression of an endogenous gene.

The invention provides for double-stranded RNAi agents capable of inhibiting the expression of a target gene in vivo. The RNAi agent may comprise a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”). The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the LINC complex inhibitor is an inhibitory nucleic acid such as miRNA, siRNA, shRNA or an antisense oligonucleotide (ASO).

In some embodiments, an inhibitory nucleic acid is a micro RNA (miRNA), or a precursor thereof (e.g. a primiRNA or a pre-miRNA). miRNA molecules have a similar structure to siRNA molecules, but are encoded endogenously, and derived from processing of short hairpin RNA molecules. They are initially expressed as long primary transcripts (pri-miRNAs), which are processed within the nucleus into 60 to 70 nucleotide hairpins (pre-miRNAs), which are further processed in the cytoplasm into smaller species that interact with RISC and target mRNA. miRNAs comprise ‘seed sequences’ that are essential for binding to target mRNA. Seed sequences usually comprise six nucleotides and are situated at positions 2 to 7 at the miRNA 5′ end.

In some embodiments, an inhibitory nucleic acid is a small interfering RNA (siRNA). As used herein, ‘siRNA’ refers to a double-stranded RNA molecule having a length between 17 to 30 (e.g. 20 to 27, e.g. ˜21 to 23) base pairs, which is capable of engaging the RNA interference (RNAi) pathway for the targeted degradation of target RNA. Double-stranded siRNA molecules may be formed as a nucleic acid complex of RNA strands having a high degree of complementarity. The strand of the double-stranded siRNA molecule having complementarity to a target nucleotide sequence (i.e. the antisense nucleic acid) may be referred to as the ‘guide’ strand, and the other strand may be referred to as the ‘passenger’ strand.

The siRNA may contain one or more overhang regions and/or capping groups at the 3′-end, 5′-end, or both ends of one or both strands e.g. comprising one or two or three nucleotides (e.g. a ‘UU’ 3′ overhang, a ‘TT’ 3′ overhang, or a ‘CCA’ 5′ overhang). The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In some embodiments, a passenger strand of an siRNA may comprise a ‘CCA’ modification at the 5′ end, i.e. the addition of nucleotides ‘CCA’. In some embodiments, a passenger strand of an siRNA according to the present disclosure may comprise a ‘TT’ modification at the 3′ end, e.g. replacing the 3′ two nucleotides.

In some embodiments, the guide strand of an siRNA according to the present disclosure may comprise or consist of an antisense nucleic acid according to an embodiment of an antisense nucleic acid described herein.

In some embodiments an siRNA according to the present disclosure may be contained within a longer shRNA sequence that undergoes processing to form the siRNA.

In some embodiments, an inhibitory nucleic acid is a short hairpin RNA (shRNA). shRNA molecules comprise sequences of nucleotides having a high degree of complementarity that associate with one another through complementary base pairing to form the stem region of the hairpin. The sequences of nucleotides having a high degree of complementarity may be linked by one or more nucleotides that form the loop region of the hairpin. shRNA molecules may be processed (e.g. via catalytic cleavage by DICER) to form siRNA or miRNA molecules. shRNA molecules may have a length of between 35 to 100 (e.g. 40 to 70) nucleotides. The stem region of the hairpin may have a length between 17 to 30 (e.g. 20 to 27, e.g. ˜21-23) base pairs. The stem region may comprise G-U pairings to stabilise the hairpin structure. An shRNA sequence described herein may comprise sequences that will be subsequently processed into shorter siRNA strand(s).

siRNA, dsiRNA, miRNAs and shRNAs for the targeted inhibition of gene and/or protein expression may be identified/designed in accordance with principles and/or using tools well known to the skilled person. Parameters and tools for designing siRNA and shRNA molecules are described e.g. in Fakhr et al., Cancer Gene Therapy (2016) 23:73-82 (hereby incorporated by reference in its entirety). Software that may be used by the skilled person for the design of such molecules is summarised in Table 1 of Fakhr et al., Cancer Gene Therapy (2016) 23:73-82, and includes e.g. siRNA Wizard (InvivoGen). Details for making such molecules can be found in the websites of commercial vendors such as Ambion, Dharmacon, GenScript, Invitrogen and OligoEngine.

In one embodiment, the LINC complex inhibitor is an inhibitor of a gene encoding a LINC complex protein (e.g. SUN1, SUN2, Nesprin-1, Nesprin-2, Nesprin-3, Nesprin-4 and/or KASH5). Such inhibitors can be designed to target any region of a nucleic acid molecule, such as a genomic or mRNA molecule, encoding the LINC complex protein. The inhibitor may hybridize to a nucleic acid molecule, such as a genomic or mRNA molecule, that encodes the LINC complex protein, leading to decreased expression of the LINC complex protein in a cell. In one embodiment, the LINC complex inhibitor is an inhibitor of SUN1. In one embodiment, the LINC complex inhibitor is an inhibitor of SUN2. In one embodiment, the LINC complex inhibitor is a combination of an inhibitor of SUN1 and an inhibitor of SUN2. In one embodiment, the LINC complex inhibitor is an inhibitor of Nesprin-1. In one embodiment, the LINC complex inhibitor is an inhibitor of Nesprin-2. In one embodiment, the LINC complex inhibitor is an inhibitor of Nesprin-3. In one embodiment, the LINC complex inhibitor is an inhibitor of Nesprin-4. In one embodiment, the LINC complex inhibitor is an inhibitor of KASH5.

In one embodiment, the LINC complex inhibitor comprises a nucleic acid sequence having at least 70% sequence identity to any one of SEQ ID NO: 44 to SEQ ID NO: 88 or a reverse complement thereof.

In one embodiment, the LINC complex inhibitor is a SUN1 inhibitor comprising a nucleic acid sequence having at least 70% sequence identity to SEQ ID NO: 101, 45, 46 or a complementary sequence thereof. The LINC complex inhibitor may comprise a nucleic acid sequence encoded by a nucleic acid sequence having at least 70% sequence identity to SEQ ID NO: 101.

In one embodiment, the LINC complex inhibitor is a SUN2 inhibitor comprising a nucleic acid sequence having at least 70% sequence identity to SEQ ID NO: 102, 59 or a complementary sequence thereof. The LINC complex inhibitor may comprise a nucleic acid sequence encoded by a nucleic acid sequence having at least 70% sequence identity to SEQ ID NO: 102.

The term at least 70% sequence identity may refer to at least 70%, 75%, 80%, 85%, 90%, 95%, 99% or 100%.

In some embodiments, to the LINC complex inhibitor may target a region of the nucleic acid encoding a LINC complex protein involved in (e.g. required for) LINC complex formation. For example, in some embodiments, the LINC complex inhibitor may target a region of a gene encoding a SUN domain-containing protein encoding a SUN domain (such as a SUN1 or SUN2 domain). In some embodiments, the LINC complex inhibitor may target a region of a gene encoding a KASH domain-containing protein encoding a KASH domain.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

In one embodiment, the LINC complex inhibitor is a site-specific nuclease (SSN) system. The SSN system may target a LINC complex protein.

SSNs capable of being engineered to generate target nucleic acid sequence-specific double strand breaks (DSBs) include zinc-finger nucleases (ZFNs), transcription activator-like effector nucleases (TALENs) and clustered regularly interspaced palindromic repeats/CRISPR-associated-9 (CRISPR/Cas9) systems.

In some embodiments the SSN system is a ZFN system, a TALEN system, CRISPR/Cas9 system, a CRISPR/Cpf1 system, a CRISPR/C2c1 system, a CRISPR/C2c2 system or a CRISPR/C2c3 system.

In some embodiments, the SSN system targets a region of the nucleic acid encoding a LINC complex protein involved in (e.g. required for) LINC complex formation. For example, in some embodiments the SSN system may disrupt expression of an exon of a gene encoding a SUN domain-containing protein encoding a SUN domain. In some embodiments the SSN system may disrupt expression of an exon of a gene encoding a KASH domain-containing protein encoding a KASH domain.

In some embodiments the SSN system is a CRISPR/Cas9 system. In such embodiments, the LINC complex inhibition may employ nucleic acid(s) encoding a CRISPR RNA (crRNA) targeting nucleic acid encoding a LINC complex protein, and a trans-activating crRNA (tracrRNA) for processing the crRNA to its mature form. CRISPR/Cas9 systems for targeted disruption of LINC complex proteins SUN1 and SUN2 are described e.g. in Schaller et al., J Virol. (2017) 91(19): pii: e00463-17, which is hereby incorporated by reference in its entirety.

Rather than expressing components of a lumenal domain of a SUN domain-containing protein or a KASH domain to disrupt a LINC complex by competing for binding with endogenous Nesprins (which comprise a KASH domain) or Sun1 and Sun2 (which comprise a SUN domain), another approach for disrupting the LINC complex is to modify the endogenous SUN domain or KASH domain so that it fails to bind to, or has reduced binding capacity for, its cognate LINC complex binding partner.

As both the SUN domain and the KASH domain are located at the C-termini of their respective proteins, one way of producing a modified SUN or KASH domain is to use a CRISPR/Cas system to modify the genes encoding SUN or KASH domain proteins to generate a premature stop codon at the 3′ end of the respective protein sequences following CRISPR-induced non-homologous end joining. This would result in a truncated protein with its C-terminal SUN or KASH domain mutated. The truncated protein would be expressed and membrane-localized, but unable to interact with its cognate LINC complex partners.

Accordingly, in some embodiments a LINC complex inhibitor is a CRISPR-Cas or other synthetic nuclease system capable of modifying nucleic acid that encodes the SUN domain or KASH domain of endogenous Sun or Nesprin protein, respectively.

In some embodiments the CRISPR-Cas system modifies the endogenous SUN domain or KASH domain of Sun1 or Nesprin-1 protein, respectively, to disrupt a LINC complex. The respective nucleic acids are Sun1 and Syne1.

In some embodiments, the CRISPR-Cas system comprises a gRNA comprising a nucleic acid sequence having at least 70% sequence identity to GCACAATAGCCTCGGATGTCG (SEQ ID NO: 23), capable of modifying the SUN domain of mouse Sun1.

In some embodiments, the CRISPR-Cas system comprises a gRNA nucleic acid targeting the human SUN1 domain. In some embodiments the gRNA comprises a nucleic acid sequence having at least 70% sequence identity to any one of SEQ ID NO:12 to SEQ ID NO: 22 as set forth in Table 1 or a reverse complement thereof.

TABLE 1

| Gene | | | | | CRISPR |
|---|---|---|---|---|---|
| Name | ENSEMBL gene ID | Sequence | SEQ ID NO: | PAM | enzyme |
| SYNE1 | ENSG00000131018 | UCGUGUAUCU GAGCAUGGGG | 1 | UGGAAU | saCas9 |
| SYNE1 | ENSG00000131018 | GCCAUUCGUG UAUCUGAGCA | 2 | UGGGGU | saCas9 |
| SYNE1 | ENSG00000131018 | UCCACCCCAU GCUCAGAUAC | 3 | ACGAAU | saCas9 |
| SYNE1 | ENSG00000131018 | GAGCAUGGGG UGGAAUGACC | 4 | GGG | spCas9 |
| SYNE1 | ENSG00000131018 | UGAGCAUGGG GUGGAAUGAC | 5 | CGG | spCas9 |
| SYNE1 | ENSG00000131018 | UCGUGUAUCU GAGCAUGGGG | 6 | UGG | spCas9 |
| SYNE1 | ENSG00000131018 | CAUUCGUGUA UCUGAGCAUG | 7 | GGG | spCas9 |
| SYNE1 | ENSG00000131018 | CCAUUCGUGU AUCUGAGCAU | 8 | GGG | spCas9 |
| SYNE1 | ENSG00000131018 | GCCAUUCGUG UAUCUGAGCA | 9 | UGG | spCas9 |
| SYNE1 | ENSG00000131018 | CCCAUGCUCA GAUACACGAA | 10 | UGG | spCas9 |
| SYNE1 | ENSG00000131018 | CCCGGUCAUU CCACCCCAUG | 11 | UUUG | Cpf1 |
| SUN1 | ENSG00000164828 | UUUUUCUAAC UGGGGCCAUC | 12 | CUGAGU | saCas9 |
| SUN1 | ENSG00000164828 | CCGAUACAGA CAGGUAUACU | 13 | CAGGAU | saCas9 |
| SUN1 | ENSG00000164828 | AACUUCGGAU UUUUUCUAAC | 14 | UGG | spCas9 |
| SUN1 | ENSG00000164828 | ACUUCGGAUU UUUUCUAACU | 15 | GGG | spCas9 |
| SUN1 | ENSG00000164828 | CUUCGGAUUU UUUCUAACUG | 16 | GGG | spCas9 |
| SUN1 | ENSG00000164828 | ACAGACAGGU AUACUCAGGA | 17 | UGG | spCas0 |
| SUN1 | ENSG00000164828 | CUGAGUAUAC CUGUCUGUAU | 18 | CGG | spCas9 |
| SUN1 | ENSG00000164828 | UUCUAACUGG GGCCAUCCUG | 19 | UUUU | Cpf1 |
| SUN1 | ENSG00000164828 | UCUAACUGGG GCCAUCCUGA | 20 | UUUU | Cpf1 |
| SUN1 | ENSG00000164828 | CUAACUGGGG CCAUCCUGAG | 21 | UUUU | Cpf1 |
| SUN1 | ENSG00000164828 | UAACUGGGGC CAUCCUGAGU | 22 | UUUC | Cpf1 |

In some embodiments, the CRISPR-Cas system comprises a gRNA comprising a nucleic acid sequence having at least 70% sequence identity to CCGTTGGTATATCTGAGCAT (SEQ ID NO: 24), capable of modifying the KASH domain of mouse Syne-1.

In some embodiments, the CRISPR-Cas system comprises a gRNA nucleic acid sequence targeting the human KASH domain set forth in SEQ ID NO:6. In some embodiments the gRNA comprises a nucleic acid sequence having at least 70% sequence identity to any one of SEQ ID NO: 1 to SEQ ID NO: 11 as set forth in Table 1 or a reverse complement thereof. In some embodiments, the CRISPR-Cas system is a CRISPR-Cas9 system or variant thereof.

In some embodiments the CRISPR-Cas system modifies the endogenous SUN domain or KASH domain of Sun2 or Nesprin-2 protein, respectively, to disrupt a LINC complex. The respective nucleic acids are Sun2 and Syne2. The guide RNA targeted sequences may be upstream of C-terminal SUN or KASH domains that are involved in forming LINC complexes. Thus CRISPR-induced deletion upstream of these regions may either result in nonsense-mediated decay of the mRNA or disrupt LINC complex interactions.

In some embodiments, the CRISPR-Cas system comprises a gRNA targeting the human SUN2 domain. In some embodiments the gRNA comprises a nucleic acid sequence having at least 70% sequence identity to any one of SEQ ID NO: 25 to SEQ ID NO: 33 as set forth in Table 2, or a reverse complement thereof.

In some embodiments, the CRISPR-Cas system comprises a gRNA targeting the human SYNE2 domain. In some embodiments the gRNA comprises a nucleic acid sequence having at least 70% sequence identity to any one of SEQ ID NO: 34 to SEQ ID NO: 42 as set forth in Table 2, or a reverse complement thereof.

TABLE 2

| | | | | | | | SEQ | | |
|---|---|---|---|---|---|---|---|---|---|
| | Gene | ENSEMBL gene | Chromo- | | | | ID | | CRISPR |
| | Name | ID | some | Position | Strand | Sequence | NO: | PAM | enzyme |
| 5' of SUN | SUN2 | ENSG00000100242 | 22 | 54331 | −1 | GCCCUGCAUCUCUGCCACAU | 25 | GGG | spCas9 |
| domain | SUN2 | ENSG00000100242 | 22 | 54328 | 1 | GAGCAAGAUCCUCACCCAUG | 26 | UGG | spCas9 |
| encoding | SUNZ | ENSG00000100242 | 22 | 54415 | 1 | AGGUGUGAUUGGAGUGACAG | 27 | AGG | spCas9 |
| region | SUN2 | ENSG00000100242 | 22 | 54218 | 1 | GUGGAAUCUCAGUUCCCGGC | 28 | CUGGAU | saCas9 |
| | SUN2 | ENSG00000100242 | 22 | 54404 | 1 | CUGCAGAAAGAAGGUGUGAU | 29 | UGGAGU | saCas9 |
| | SUN2 | ENSG00000100242 | 22 | 54327 | −1 | UGCAUCUCUGCCACAUGGGU | 30 | GAGGAU | saCas9 |
| | SUN2 | ENSG00000100242 | 22 | 51836 | 1 | CGCAGGGAAACUGCUGCUCG | 31 | ULUC | Cpf1 |
| | SUN2 | ENSG00000100242 | 23 | 53114 | −1 | CCACUCUGACUUCAGCUGCU | 32 | UUUG | Cpf1 |
| | SUNZ | ENSG00000100242 | 22 | 54373 | −1 | UGCAGCGUICAGGCUCAGGGA | 33 | DULC | Cof1 |
| 5' of | SYNE2 | ENSG00000054654 | 12 | 64225505 | 1 | CCCAUGCUGAGGUACACCAA | 34 | UGG | spCas9 |
| KASH | SYNE2 | ENSG00000054654 | 12 | 64225438 | −1 | GCAGCUGUAGUCUUCUUCGG | 35 | AGG | spCas9 |
| C- | SYNE2 | ENSG00000054654 | 12 | 64225493 | −1 | CAUUGGUGUACCUCAGCAUG | 36 | GGG | spCas9 |
| terminal | SYNE2 | ENSG00000054654 | 12 | 64225464 | −1 | GACCGGGCAAAGUUGUUGGC | 37 | CUGAGU | saCas9 |
| tetra- | SYNEZ | ENSG00000054654 | 12 | 64225495 | −1 | CCCAUUGGUGUACCUCAGCA | 35 | UGGGGU | saCas9 |
| peptide | SYNE2 | ENSG00000054654 | 12 | 64225388 | −1 | GCAGCAGGAGGAGCAGCUGC | 39 | AGGGGU | saCas9 |
| motif | SYNE2 | ENSG00000054654 | 12 | 64225493 | 1 | CCCGGUCCUUUUACCCCAUG | 40 | UUUG | Cpf1 |
| | SYNEZ | ENSG00000054654 | 12 | 64225505 | 1 | ACCCCAUGCUGAGGUACACC | 41 | UUUU | Cof1 |
| | SYNE2 | ENSG00000054654 | 12 | 64225506 | 1 | CCCCAUGCUGAGGUACACCA | 42 | ULLA | Cpf1 |

In one embodiment, the LINC complex inhibitor is a nucleic acid construct comprising a nucleic acid sequence encoding a CRISPR nuclease and a nucleic acid sequence encoding a guide RNA.

In one embodiment, the LINC complex inhibitor is an SSN system comprising a CRISPR nuclease protein and a guide RNA.

In one embodiment, the LINC complex inhibitor comprises a nucleic acid sequence having at least 70% sequence identity to CCAUCCUGAGUAUACCUGUCUGUAU (SEQ ID NO: 43) or a reverse complement thereof.

In one embodiment, the LINC complex inhibitor comprises a nucleic acid sequence having at least 70% sequence identity to any one of SEQ ID NO: 44 to SEQ ID NO: 88 or a reverse complement thereof.

TABLE 3

| | Gene | Gene | | SEQ |
|---|---|---|---|---|
| Taxon | ID | Symbol | Target Seq | ID NO: |
| human | 23353 | SUN1 | UUCAUGGACGAGGGCAUAUAC | 44 |
| human | 23353 | SUN1 | GCUGUUCUGAAACUUACGAAA | 45 |
| human | 23353 | SUN1 | GCUGUUCUGAAACUUACGAAA | 46 |
| human | 23353 | SUN1 | CAGAUACACUGCAUCAUCUUU | 47 |
| human | 23353 | SUN1 | CCAGAUACACUGCAUCAUCUU | 48 |
| human | 23353 | SUN1 | GCAGAGGUUCUCAUCACAGUU | 49 |
| human | 23353 | SUN1 | GAGCACAAAUUGGACCCUGUA | 50 |
| human | 23353 | SUN1 | GAACUAGAACAGACCAAGCAA | 51 |
| human | 23353 | SUN1 | GAACUAGAACAGACCAAGCAA | 52 |
| human | 23353 | SUN1 | GCUUUCCAAAUAGUGGAACUU | 53 |
| human | 23353 | SUN1 | GCUUUCCAAAUAGUGGAACUU | 54 |
| human | 23353 | SUN1 | CCUCCCAAAGUACUGGGAUUA | 55 |
| human | 25777 | SUN2 | CGAGCCUAUUCAGACGUUUCA | 56 |
| human | 25777 | SUN2 | GUGUAUAUAUGUAGCAUAUCA | 57 |
| human | 25777 | SUN2 | GAGGAAAUCCAGCAACAUGAA | 58 |
| human | 25777 | SUN2 | GCCUAUUCAGACGUUUCACUU | 59 |
| human | 25777 | SUN2 | CUGCAGAAAGAAGGUGUGAUU | 60 |
| human | 25777 | SUN2 | GCAAGACUCAGAAGACCUCUU | 61 |
| human | 25777 | SUN2 | GAAGUCAGAGUGGCAAAGCAU | 62 |
| human | 25777 | SUN2 | GCUCUGACUUCUAGGAGCACA | 63 |
| human | 25777 | SUN2 | UGCUUAGCUCCAGAGCAGGAU | 64 |
| human | 23345 | SYNE1 | GCGUAGUGAUAAGACUGAUUU | 65 |
| human | 23345 | SYNE1 | CAGAAGUGCUGGUAGCAUAAG | 66 |
| human | 23345 | SYNE1 | GCGUGUGGAAAGAUACCAUAA | 67 |
| human | 23345 | SYNE1 | GGCCAAACCUGGGCAAAUUUA | 68 |
| human | 23345 | SYNE1 | GCGUUCUUGGGAAACCUUAAA | 69 |
| human | 23345 | SYNE1 | AGCCGUGCAGCAGCUUGAUAA | 70 |
| human | 23345 | SYNE1 | CAAUCAGGCAGCAUCACAUUU | 71 |
| human | 23345 | SYNE1 | GAACCAGUCCGUGCUACAAAU | 72 |
| human | 23345 | SYNE1 | GGCGAAUGCAGUAGCAGUAUA | 73 |
| human | 23345 | SYNE1 | GCAAUCCCAAACUACCAACAA | 74 |
| human | 23345 | SYNE1 | GCAGUUUAACUCAGACUUGAA | 75 |
| human | 23345 | SYNE1 | GCUGUGGUACAAUAUGAACAA | 76 |
| human | 23345 | SYNE1 | GAUUCCCUGGAGAAACUACAU | 77 |
| human | 23224 | SYNE2 | AUUGAACGAGAGGCAAUUAUU | 78 |
| human | 23224 | SYNE2 | CAUCGACGAUCUCCAUAUUUC | 79 |
| human | 23224 | SYNE2 | GAUGGAAACAAUCAAUCAUAU | 80 |
| human | 23224 | SYNE2 | CGGACAUUAGUAUUGAAGAAA | 81 |
| human | 23224 | SYNE2 | GCAGUGAAAUACCUCUUGAAU | 82 |
| human | 23224 | SYNE2 | CCUCAGUUAUAUCCGACCUAU | 83 |
| human | 23224 | SYNE2 | CCUCAGUUAUAUCCGACCUAU | 84 |
| human | 23224 | SYNE2 | GCCACCUAUGAGUCUGUCAAU | 85 |
| human | 23224 | SYNE2 | GCGCUAUGAAAGAACGGAGUU | 86 |
| human | 23224 | SYNE2 | GCGCUAUGAAAGAACGGAGUU | 87 |
| human | 23224 | SYNE2 | GGCAGGAGAAUUGCUUGAAUC | 88 |

In one embodiment, the LINC complex inhibitor is a dominant negative SUN construct. The dominant negative SUN construct may comprise an amino acid sequence having at least 70% sequence identity to SEQ ID NO: 106.

The term “at least 70% sequence identity” may refer to at least 70%, 80%, 90%, 95% or more sequence identity as compared to a reference sequence.

In one embodiment, contacting the cell with a LINC complex inhibitor leads to inhibition of durotaxis. The inhibition of durotaxis may lead to a decrease in the durotactic index. The decrease may be, for example, a decrease of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or up to and including a 100% decrease or any decrease between 5-100% as compared to a reference level.

In one embodiment, the cell that is to be targeted is a mammalian cell. The cell may, for example, be a myofibroblast, an immune cell or a cancer cell (e.g. a metastatic cancer cell such as a metastatic breast cancer cell). The immune cell may be a lymphocyte (e.g. T cell, B cell, or NK cell), neutrophil or monocyte/macrophage.

In one embodiment, there is provided a Linker of Nucleoskeleton and Cytoskeleton (LINC) complex inhibitor for use of inhibiting durotaxis in a cell.

In one embodiment, there is provided the use of a Linker of Nucleoskeleton and Cytoskeleton (LINC) complex inhibitor in the manufacture of a medicament for inhibiting durotaxis in a cell.

Provided herein is a nucleic acid molecule encoding a LINC complex inhibitor as defined herein.

The term “encoding” or “encodes” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e. rRNA, tRNA and mRNA) or a defined sequence of amino acids and the biological properties resulting therefrom. Thus, a gene encodes a protein if transcription of a gene and translation of mRNA corresponding to that gene produces the protein in a cell or other biological system. Both the coding strand and the non-coding strand, used as the template for transcription of a gene or cDNA, can be referred to as encoding the protein or other product of that gene or cDNA.

Provided herein is a construct comprising a nucleic acid molecule as defined herein.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors.

The term “expression” refers to the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

The nucleic acid may be, or may be comprised in, a vector. A “vector” as used herein may be a nucleic acid used as a vehicle to transfer exogenous nucleic acid into a cell. The vector may be a vector for expression of the nucleic acid in the target cell. Such vectors may include a promoter sequence operably linked to the nucleic acid sequence to be expressed. A vector may also include a termination codon and expression enhancers. In this specification the term “operably linked” may include the situation where a selected nucleic acid sequence and regulatory nucleic acid sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. Where appropriate, the resulting transcript may then be translated into a desired polypeptide.

Any suitable vectors, promoters, enhancers and termination codons known in the art may be used. Suitable vectors include viral vectors, e.g. retroviral vectors, lentiviral vectors, adenovirus vectors, adeno-associated virus vectors, vaccinia virus vectors and herpesvirus vectors, transposon-based vectors, and artificial chromosomes (e.g. yeast artificial chromosomes), e.g. as described in Maus et al., Annu Rev Immunol (2014) 32:189-225 or Morgan and Boyerinas, Biomedicines 20164, 9, which are both hereby incorporated by reference in its entirety.

In some embodiments, a vector may be an adeno-associated viral vector. In some embodiments, a vector may be an adeno-associated viral vector of one of the following serotypes: AAV6 or AAV6.2FF (i.e. lung-tropic AAVs), or AAV8, AAV-LKO3 (i.e. liver-tropic AAVs),

In some embodiments, the vector is a lipid nanoparticle (LNP) for delivering the LINC complex inhibitor to a cell.

As used herein, the term “lipid nanoparticle” refers to a nanoparticle made from lipids (e.g., a cationic or ionisable lipid, a non-cationic lipid, a conjugated lipid and cholesterol), wherein the nucleic acid is fully encapsulated within the lipid. LNPs may contain multiple lipid layers as well as microdomains of lipids and nucleic acids and may thus be distinguished from lipoplexes (in which the nucleic acid is not encapsulated), micelles (which only contain a lipid monolayer) and liposomes (which only contain a lipid bilayer).

### Composition

Provided herein is a pharmaceutical composition comprising a LINC complex inhibitor as described herein, and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, colouring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. In some embodiments, the administration is parenteral (e.g., intravenous, subcutaneous, intraperitoneal, intramuscular, intranasal, topical or transdermal).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

The pharmaceutical composition of the present invention may be suitable for topical administration to the skin may comprise inhibitors dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the pharmaceutical composition of the invention.

### Treatment

Disclosed herein is a method of preventing or treating fibrosis or a disease associated with fibrosis in a subject, the method comprising administering an effective amount of a LINC complex inhibitor to the subject.

In one embodiment, there is provided a LINC complex inhibitor for use in preventing or treating fibrosis or a diseases associated with fibrosis in a subject.

In one embodiment, there is provided the use of a LINC complex inhibitor in the manufacture of a medicament for preventing or treating fibrosis or a diseases associated with fibrosis in a subject.

The terms “treating”, “treatment” and the like also include relieving, reducing, alleviating, ameliorating or otherwise inhibiting the effects of the condition for at least a period of time. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The term “administering” refers to contacting, applying, injecting, transfusing or providing an inhibitor as referred to herein to a subject.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In one embodiment, the subject is a human subject.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

In one embodiment, the LINC complex inhibitor is combined with a pharmaceutically acceptable carrier to provide pharmaceutical compositions for administering to a subject.

In various embodiments of the composition, the carrier comprises one or more components selected from the group consisting of a saline solution, a sugar solution, a polymer, a peptide, a lipid, a cream, a gel, a micellar material, a silica nanoparticle, a plasmid, and a viral vector. Other carriers include one or more of the following: a polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer, biodegradable polyesters, such as poly(lactic acid) (PLA), poly(glycolic acid) (PGA), and poly(lactic-co-glycolic acid) (PLGA), and polyamidoamine (PAMAM) dendrimers.

In one embodiment, the LINC complex inhibitor treats or prevent a disease associated with fibrosis. The LINC complex inhibitor may treat or prevent fibrosis.

As used herein, “fibrosis” refers to the formation of excess fibrous connective tissue as a result of the excess deposition of extracellular matrix components, for example collagen. Fibrous connective tissue is characterized by having extracellular matrix (ECM) with a high collagen content. The collagen may be provided in strands or fibers, which may be arranged irregularly or aligned. The ECM of fibrous connective tissue may also include glycosaminoglycans.

As used herein, “excess fibrous connective tissue” refers to an amount of connective tissue at a given location (e.g. a given tissue or organ, or part of a given tissue or organ) which is greater than the amount of connective tissue present at that location in the absence of fibrosis, e.g. under normal, non-pathological conditions. As used herein, “excess deposition of ECM components” refers to a level of deposition of one or more ECM components which is greater than the level of deposition in the absence of fibrosis, e.g. under normal, non-pathological conditions.

A disease associated with or characterized by fibrosis refers to a disease in which fibrosis and/or profibrotic processes are pathologically implicated. A “disease characterized by fibrosis” may be fibrosis, e.g. of any cell, tissue or organ.

Diseases associated with or characterized by fibrosis include but are not limited to: respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, cirrhosis, non-alcoholic fatty liver disease (NAFLD), non-alcoholic steatohepatitis (NASH), primary biliary cirrhosis (PBC), schistosomal liver disease, cardiovascular conditions such as hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Crohn's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's opthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

In one embodiment, the disease associated with fibrosis is skin, liver or lung fibrosis.

The disease associated with fibrosis might be skin fibrosis, systemic sclerosis, systemic scleroderma, hypertrophic scarring, keloids, renal fibrosis, cardiac fibrosis, primary sclerosing cholangitis or primary biliary cholangitis.

In one embodiment, the method comprises inhibiting TGFβ activity in the subject. The method may comprise inhibiting TGFβ-mediated increase in smooth muscle actin in a cell. The cell may be, for example, dermal fibroblast, pulmonary fibroblast, or hepatic stellate cells.

In one embodiment, there is provided a method of preventing, inhibiting or treating steatosis in a liver of a subject, the method comprising administering an effective amount of a LINC complex inhibitor to the subject.

Disclosed herein is a method of inhibiting or preventing metastasis of a cancer in a subject, the method comprising administering an effective amount of a LINC complex inhibitor to the subject.

In one embodiment, there is provided a LINC complex inhibitor for use in inhibiting or preventing metastasis of a cancer in a subject.

In one embodiment, there is provided the use of a LINC complex inhibitor in the manufacture of a medicament for inhibiting or preventing metastasis of a cancer in a subject.

A cancer may be any cancer. As used herein, cancers include any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Disclosed herein is a method of treating a metastatic cancer in a subject, the method comprising administering an effective amount of a LINC complex inhibitor to the subject.

In one embodiment, there is provided a LINC complex inhibitor for use in treating a metastatic cancer in a subject.

In one embodiment, there is provided the use of a LINC complex inhibitor in the manufacture of a medicament for treating a metastatic cancer in a subject.

The subject may be further administered a cancer therapy.

Disclosed herein is a method of treating or preventing a disease associated with inflammation in a subject, the method comprising administering an effective amount of a LINC complex inhibitor to the subject.

In one embodiment, there is provided a LINC complex inhibitor for use in treating or preventing a disease associated with inflammation in a subject.

In one embodiment, there is provided the use of a LINC complex inhibitor in the manufacture of a medicament for treating or preventing a disease associated with inflammation in a subject.

The LINC complex inhibitor may treat or prevent the disease associated with inflammation by inhibiting durotaxis in the subject.′

Disclosed herein is a method of promoting wound healing or tissue regeneration in a subject, the method comprises administering an effective amount of a LINC complex inhibitor to the subject.

By wound is meant an injury to any tissue, including for example, acute, subacute, delayed or difficult to heal wounds, and chronic wounds. The wound may be present on an external skin surface of a subject. Examples of wounds may include both open and closed wounds. Wounds include, for example, burns, incisions, excisions, lacerations, abrasions, puncture or penetrating wounds, surgical wounds, contusions, hematomas, crushing injuries, and ulcers.

In one embodiment, there is provided a LINC complex inhibitor for use in promoting wound healing or tissue regeneration in a subject.

In one embodiment, there is provided the use of a LINC complex inhibitor in the manufacture of a medicament for promoting wound healing or tissue regeneration in a subject.

In one embodiment, the LINC complex inhibitor treats or prevents scarring.

Disclosed herein is a method of treating a wound in a subject, the method comprises administering an effective amount of a LINC complex inhibitor to the subject.

In one embodiment, there is provided a LINC complex inhibitor for use in treating a wound in a subject.

In one embodiment, there is provided the use of a LINC complex inhibitor in the manufacture of a medicament for treating a wound in a subject.

In one embodiment, the method comprises topically administering the LINC complex inhibitor to the subject.

In one embodiment, there is provided a method inhibiting TGFβ activity in a cell or tissue of a subject, the method comprising administering an effective amount of a LINC complex inhibitor to the subject.

The subject may be suffering from a condition or disease that is associated with elevated or abnormal TGFβ activity. The condition or disease may, for example, be a disease associated with or characterized by fibrosis, cancer, tuberculosis, heart disease, Marfan syndrome, Loeys-Dietz syndrome, obesity, diabetes, multiple sclerosis or Alzheimer's disease.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Example 1

### Materials and Methods

Fabrication of Substrate with Pillar Array with a Stepped Jump in Stiffness

The pillar array with a stepped jump in stiffness was fabricated by the stepwise fabrication method. First, the mixed PDMS (polydimethylsiloxane) with desired mixing ratio of PDMS base:curing agent was prepared. Then coumarin-343 dissolved in ethanol (1 mM) was mixed with PDMS mixture in 1:10 (w/w) ratio. The PDMS and dye mixture was poured onto half of a silicon mould and spin coated. The spin-coated silicon mould was cured at 80° C. for 4 hours. After fabrication of the first layer, the PDMS with different mixing ratio to the first layer were poured the other half of the mould, spin-coated and cured for 4 hours. Then the cured monolayer was peeled off from the mould and attached to the 35 mm glass bottom dish (no. 0 thickness, MatTek). The uniformity of a pillar structure around the stiffness interface was confirmed using fluorescence, DIC, AFM and SEM images. The stiffness of fabricated pillars, k, was calculated by Euler-Bernoulli's beam-theory:

$$k = \frac{3}{4} π E \frac{{r}^{4}}{{L}^{3}}$$

where r and L are the radius and height of the pillar, respectively, and E is the Young's modulus of the PDMS that determined by the mixing ratio of PDMS base:curing agent⁴⁴. In this paper, the mixing ratio of PDMS varies from 2:1 to 15:1. The fabricated pillar array was coated with 10 μg ml-1 of fibronectin for 1 h, at 37° C. before cell seeding.

### Scanning Electron Microscopy and Atomic Force Microscopy

The stiffness interfaces of the pillar array were imaged in high-magnification by scanning electron microscopy (SEM) and atomic force microscopy (AFM). A pillar array with a stiffness interface was cut into 1×2 cm and attached to the bulk PDMS block. For the SEM imaging, the surface of samples was coated with gold using an ion sputtering machine (Seron technologies) for 15 min and imaging was carried under 15.0 kV using JSM-7600F (Jeol). AFM imaging was done using XE-7 (Park systems) and PPP-CONTSCR cantilever (0.2 N/m of force constant and 25 kHz of resonance frequency). Images were acquired under the non-contact mode.

### Microscopy

Time-lapse and snapshot live-cell imaging were conducted by Eclipse Ti2-E inverted microscope (Nikon), equipped with CSU-W1 spinning disk confocal unit (Yokogawa) and ORCA-Fusion camera (Hamamatsu) that controlled by the MetaMorph software (Molecular Devices). Cells were kept in the chamber maintaining 37° C., 5% CO₂ during imaging. 60×, 1.2 numerical aperture (NA) water immersion objective lens was used for the imaging of pillars and fluorescence imaging and ×10, 0.45 NA air objective lens was used for live-cell imaging of cells for durotaxis ratio. Epifluorescence imaging was performed on an Olympus IX-83 microscope with a 40×, 0.6 NA air objective, and a Hamamatsu ORCA flash 4 camera.

### Measuring Deflection of Pillars and Traction Force

DIC images of pillars were captured using ×60 1.2 NA, water immersion objective lens, and for time-lapse images, images were taken every 10 min. Acquired images were processed using ImageJ (National Institute of Health) and MBI pillar tracker plugin. Briefly, this plugin uses preset settings of pillars (diameter, spacing, arrangement, angle of the grid, and 2-D parameterized Gaussian sigma) to detect the center of a pillar before and after its deflection using Levenberg-Marquardt algorithm. The noise of the measurement was tested by measuring the deflection of pillars when pillars are not under the cell and the noise was less than 30 nm. Traction force was calculated by multiplying the deflection by the stiffness of the pillar according to the Hooke's Law, F=k×x.

### Tracking Nucleus and Centroid of the Cell

Cell centroid was obtained by computing the center of mass of the segmentation mask image of the cell and implemented using MATLAB function regionprops. Nuclear centroid was obtained using Discriminative Correlation Filter with Channel and Spatial Reliability (DCF-CSR) object tracking algorithm⁴⁶, which tracks the nucleus using a rectangular region of interest bounding box. Since the nucleus's shape remains a regularly shaped circle or ellipse, the center of the bounding box matches the center of the nucleus. The tracker was implemented using OpenCV TrackerCSRT in Python. The results were visualized and examined for every frame, and manual correction were applied for frames where tracking was unsatisfactory to ensure accuracy of results.

### Cell Culture

Mice were maintained at the A*STAR Biological Resource Centre facility in accordance with the guidelines of the Institutional Animal Care and Use Committee. Fibroblasts were obtained from E18.5 mouse embryos by digestion of muscle tissue in enzyme solution (0.5% w/v collagenase type II, 1.2 U/ml dispase, 2.5 mM CaCl2, 25 mM HEPES in Hanks' Balanced Salt Solution) for 30 min at 37° C. with gentle trituration using a plastic pipette tip every 10-15 min. Following addition of DMEM (Sigma), containing 10% FBS, resultant tissue slurry was filtered successively through 70 mm and 40 mm cell strainers. Cells were plated onto tissue culture plates for 1-2 hr in 37° C. and 5% CO2 to allow fibroblasts to adhere. Fibroblasts were spontaneously immortalized by continuous passaging and were maintained at 37° C., 5% CO₂ incubator with DMEM containing 10% of heat-inactivated fetal bovine serum (FBS) and 100 IU ml⁻¹ penicillin-streptomycin. Mycoplasma contamination test were conducted and found negative. For the live-cell imaging, cells were seeded over the fibronectin-coated pillar array and incubated at least 1 h to spread. For the SUN1 dominant-negative cell lines, cells were cultured using the same DMEM, and treated with 1 μgml⁻¹ of doxycycline at least 24 h before experimentation in order to disrupt the LINC complex.

### Lentivirus Transduction

HA-DNSun1 was PCR amplified from pcDNA3.1SS-HA-Sun1L-KDEL and cloned into an inducible lentiviral vector, pTripZ (Open Biosystem). F-tractin (GenBank: NM_031045.2) tagged with mScarlet-I (GenBank: APD7653) was used to label actin and vinculin tagged with mNeonGreen (GenBank: KC295282.1) was used to monitor focal adhesions. To generate a viral vector encoding F-tractin and mScarlet-I, Ftractin and mScarlet-I were ordered as gene blocks (Integrated DNA Technologies) and amplified with PCR (F-tractin primers: forward 5-aaaaaggatccaccatgggcatggaacatg-3′ (SEQ ID NO: 89), reverse 5′-aaaaagaattctactggtgggtccgatcctgctgcag-3′ (SEQ ID NO: 90); mScarlet-I primers: forward 5′-aaaaagaattcatgagtaaaggagaagctgtgattaaag-3′ (SEQ ID NO: 91), reverse 5′-aaaaagaattcttatttgtatagttcatccatgccaccgg-3′ (SEQ ID NO: 92)). The F-tractin and mScarlet-I PCR products were digested with BamHI/EcoRI and EcoRI respectively. The digested fragments were then ligated. pLV-EF1a-IRES_Blast (Addgene) was cut with BamHI and EcoRI and ligated with the previously ligated F-tractin-mScarlet-I fragment to obtain pLV-EF1a-Ftractin-mScarlet-I. F-tractin-mScarlet-I insert integrity was verified by sequencing. mNeonGreen-Vinculin was synthesized and cloned into pTwist Lenti SFFV puro WPRE (Twist Bioscience) to obtain pTwist-Lenti-SFFV-mNeonGreen-Vinculin and insert integrity was verified by sequencing. Lentivirus particles were generated in 293T cells using pTripZ-HA-DNSun1, pLV-EF1a-Ftractin-mScarlet-I or pTwist-Lenti-SFFV-mNeonGreen-Vinculin and viral supernatant concentrated using polyethylene glycol (PEG) 8000 (Merck) solution. pTripZ-HA-DNSun1 viral particles were used to transduce immortalized fibroblasts to generate stable fibroblast cell lines harboring a transgene for doxycycline-inducible expression of HA-DNSun1 following puromycin selection at 1 μml⁻¹. LINC complex disruption was achieved in this stable fibroblast cell line by inducing expression of HA-DNSun1 using 1 μgml⁻¹ doxycycline. Immortalized wild type and HA-DNSun1 fibroblasts were transduced with pLV-EF1a-Ftractin-mScarlet-I or pTwist-Lenti-SFFV-mNeonGreen-Vinculin virus particles and incubated for 3 to 7 days with 10 μgml⁻¹ of puromycin and blasticidin (Sigma). Transduced cells were sorted by FACS after fluorescence signals from the tagged-F-tractin and vinculin were observed.

HA-DNSUN1 sequence

| | |
|---|---|
| HA-DNSUN1 | ATGAAGTGGGTAACCTTTATTTCCCTTCTTTTTCTCTTTAGCTCGGCCTATTCC |
| DNA sequence | AGGGGTGTGTTTCGTCGAGATGGGCCCGCTTACCCTTACGATGTACCGGATT |
| | ACGCACTCGACGATTCCAAGGGCATGCATAGACCTGGCCCTCTTCCCCCGAG |
| | CCCACCTCCAAAGGTTGATCACAAGGCTTCCCAGTGGCCTCAGGAGAGTGAC |
| | ATGGGGCAGAAGGTAGCTTCTTTGAGTGCGCAGTGCCACAACCATGATGAG |
| | AGACTTGCAGAGCTGACAGTCCTGCTTCAGAAACTACAGATACGGGTAGACC |
| | AAGTGGATGACGGCAGGGAAGGGCTGTCACTGTGGGTCAAGAATGTGGTTG |
| | GACAGCACCTGCAGGAGATGGGCACCATAGAACCACCTGATGCTAAGACTG |
| | ACTTCATGACTTTCCACCATGACCATGAAGTGCGTCTCTCCAACTTGGAAGA |
| | TGTTCTTAGAAAACTGACAGAAAAATCTGAGGCTATCCAGAAGGAGCTGGA |
| | AGAAACCAAGCTGAAAGCAGGCAGCAGGGATGAAGAGCAGCCCCTCCTTGA |
| | CCGTGTGCAGCACCTAGAACTGGAACTGAACCTGTTGAAGTCACAGCTGTCA |
| | GACTGGCAGCATCTGAAGACCAGCTGTGAGCAGGCTGGGGCCCGCATCCAG |
| | GAGACTGTGCAGCTCATGTTCTCTGAGGATCAGCAGGGCGGTTCCCTCGAGT |
| | GGCTATTAGAGAAGCTTTCTTCTCGGTTCGTGAGCAAGGATGAGCTGCAGGT |
| | GCTCTTACATGACCTTGAGCTGAAACTGCTGCAGAATATCACACACCACATC |
| | ACCGTGACAGGACAGGCCCCGACATCCGAGGCTATTGTGTCTGCCGTGAATC |
| | AGGCAGGGATTTCAGGAATCACAGAAGCGCAAGCACATATCATTGTGAACA |
| | ATGCTCTGAAGCTGTACTCCCAAGACAAGACGGGGATGGTGGACTTTGCTCT |
| | GGAGTCTGGAGGTGGCAGCATCCTAAGCACTCGGTGCTCTGAGACCTATGAG |
| | ACCAAGACGGCACTGCTGAGCCTGTTTGGGGTCCCACTGTGGTACTTCTCAC |
| | AGTCACCTCGAGTGGTGATCCAGCCCGACATCTACCCAGGGAATTGCTGGGC |
| | GTTCAAAGGTTCCCAGGGGTACCTGGTGGTGCGGTTGTCCATGAAGATCTAC |
| | CCAACCACATTCACCATGGAACACATTCCAAAGACACTATCACCCACTGGTA |
| | ACATCTCCAGTGCCCCCAAAGACTTTGCAGTCTATGGACTGGAAACGGAGTA |
| | TCAAGAAGAGGGGCAGCCTCTGGGACGGTTCACCTATGACCAGGAAGGAGA |
| | CTCACTCCAGATGTTCCACACACTGGAAAGACCTGACCAAGCCTTCCAGATA |
| | GTAGAGCTCCGGGTCCTGTCCAACTGGGGCCACCCTGAGTACACTTGCCTCT |
| | ACCGGTTCCGAGTCCACGGAGAGCCCATCCAGAAAGATGAGTTGTAG (SEQ |
| | ID NO: 93) |
| HA-DNSUN1 | MKWVTFISLLFLFSSAYSRGVFRRDGPAYPYDVPDYALDDSKGMHRPGPLPPSP |
| protein sequence | PPKVDHKASQWPQESDMGQKVASLSAQCHNHDERLAELTVLLQKLQIRVDQV |
| | DDGREGLSLWVKNVVGQHLQEMGTIEPPDAKTDFMTFHHDHEVRLSNLEDVLR |
| | KLTEKSEAIQKELEETKLKAGSRDEEQPLLDRVQHLELELNLLKSQLSDWQHLK |
| | TSCEQAGARIQETVQLMFSEDQQGGSLEWLLEKLSSRFVSKDELQVLLHDLELK |
| | LLQNITHHITVTGQAPTSEAIVSAVNQAGISGITEAQAHIIVNNALKLYSQDKTGM |
| | VDFALESGGGSILSTRCSETYETKTALLSLFGVPLWYFSQSPRVVIQPDIYPGNCW |
| | AFKGSQGYLVVRLSMKIYPTTFTMEHIPKTLSPTGNISSAPKDFAVYGLETEYQE |
| | EGQPLGRFTYDQEGDSLQMFHTLERPDQAFQIVELRVLSNWGHPEYTCLYRFRV |
| | HGEPIQKDEL (SEQ ID NO: 94) |
| Signal sequence | MKWVTFISLLFLFSSAYSRGVFRRD (SEQ ID NO: 95) |
| from human | |
| serum albumin | |
| HA tag | YPYDVPDYA (SEQ ID NO: 96) |
| Mouse Sun1 458- | DDSKGMHRPGPLPPSPPPKVDHKASQWPQESDMGQKVASLSAQCHNHDERLAE |
| 913 amino acids | LTVLLQKLQIRVDQVDDGREGLSLWVKNVVGQHLQEMGTIEPPDAKTDFMTFH |
| | HDHEVRLSNLEDVLRKLTEKSEAIQKELEETKLKAGSRDEEQPLLDRVQHLELE |
| | LNLLKSQLSDWQHLKTSCEQAGARIQETVQLMFSEDQQGGSLEWLLEKLSSRFV |
| | SKDELQVLLHDLELKLLQNITHHITVTGQAPTSEAIVSAVNQAGISGITEAQAHII |
| | VNNALKLYSQDKTGMVDFALESGGGSILSTRCSETYETKTALLSLFGVPLWYFS |
| | QSPRVVIQPDIYPGNCWAFKGSQGYLVVRLSMKIYPTTFTMEHIPKTLSPTGNISS |
| | APKDFAVYGLETEYQEEGQPLGRFTYDQEGDSLQMFHTLERPDQAFQIVELRVL |
| | SNWGHPEYTCLYRFRVHGEPIQ (SEQ ID NO: 97) |

### Mouse Models

Mice were maintained at the A*STAR Biological Resource Centre facility in accordance with the guidelines of the Institutional Animal Care and Use Committee. Sun1 “knockout first, conditional ready” mice (Sul1^{tm1a(EUCOMM)Wtsi}) were obtained from the International Mouse Phenotyping Consortium, which were crossed to β-actin-Flp mice to obtain conditional knockout Sun1^{tm1c(EUCOMM)Wtsi} mice (Sun1^{flax}). Sun2 null mice (Sun2^{-/-}) and Rosa26 tamoxifen inducible Cre mice (Gt(ROSA)26Sor^{tm1(cre/ERT)Brn} or Rosa26^{MerCreMer}) were previously published. Sun2^{-/-} mice were crossed with Sun1^{flox/flox} mice to obtain Sun1^{+/flox};Sun2^{+/-} mice, which were then backcrosseed at least 5 times into Sun1^{flox/flox} homozygous mice. Since Sun1^{flox/flox} mice are of C57/B6 genetic background, Sun1^{+/flox};Sun2^{+/-} of C57/B6 background were obtained. Rosa26^{MerCreMer} mice were previously backcrossed into C57/B6 genetic background. Sun1^{+/flox};Sun2^{+/-} were crossed with Rosa26^{MerCreMer/MerCreMer} mice to obtain Sun1^{+/flox};Sun2^{+/-};Rosa26^{+/MerCreMer} mice, which were then intercrossed to obtain Sun1^{flox/flox};Sun2^{+/+};Rosa26^{MerCreMer/MerCreMer} and Sun1^{flox/flox};Sun2^{-/-};Rosa26^{MerCreMer/MerCreMer} mice. To ablate the LINC complex in vivo, a mouse line harbouring conditional alleles for Sun1 (Sun1^{tm1c/tm1c}), null alleles for Sun2 (Sun2^{-/-}), and a transgene for tamoxifen-regulated Cre recombinase at the safe harbour Rosa26 locus was established. To ablate only Sun1 in vivo, Sun1^{flox/flox};Sun2^{+/+};Rosa26^{MerCreMer/MerCreMer} mice were used. 2 weeks prior to commencement of bleomycin injury studies, mice were injected with corn oil as a vehicle control, or with tamoxifen at a dose of 50 mg/kg body weight once daily over 3 days to induce Sun1 recombination. For the bleomycin (BLM) injury studies, 7-9 week old mice were used. At day 0, Bleomycin (80 mU/kg) was used to induce lung fibrosis. Mice were anaesthetized using 2% isoflurane. Once the animal was stabilized, it was placed on a platform, and two front upper teeth were tied to the platform using a rubber band, with the platform adjusted to a ˜60° angle. The mouth was opened carefully and tongue was pulled out gently using blunt forceps to locate the trachea. A lamp light source was used to identify the tracheal epiglottis movement by carefully pushing the jaw to the front using a small metal laryngoscope. Simultaneously, with blunt forceps, the tongue was gently pulled out of the mouth to get a clear view of the trachea. 50 μl BLM solution was administered gently using a blunt cannulation tube attached to a syringe, and inserted into the trachea. Once the procedure was completed, the animal was removed from the anesthetic unit and placed on another platform. The mouse was then rolled left and right to facilitate insult of BLM to both left and right lungs. Animals were returned to their cages and for the next 48 h, their activity and health status was checked and body weight was recorded daily. At day 21, animals were sacrificed. Before euthanasia, blood was collected from the retro orbital plexus. Broncho Alveolar Lavage (BAL) was performed to collect lung fluid. Following dissection, the left lung was taken for histopathological analysis and right lung for hydroxyproline and other analysis. Fibrotic scores were determined using the method of Ashcroft (1988).

### Immunological Assays

On the terminal day of the BLM study (day 21), Bronchoalveolar lavage (BAL) was performed. The procedure was done three times in one animal and each time 0.4 ml of PBS was administered and withdrawn to obtain ˜1 to 1.2 ml of lung fluid. Total cell count in BAL fluid was determined using an automatic cell counter or manual counting using a Neubauer chamber. The remaining BAL fluid was centrifuged at 10000 rpm for 10 min, supernatants were collected and stored at −80 C for cytokine analysis and cell pellets were used for counting of immune cells. The cell pellet was mixed with normal mouse serum. The resulting cell suspension was then spread on clean slides to make a smear. Slide dried overnight were stained using Leishman's stain for differential counts of neutrophils, lymphocytes, and macrophages. By using the total cell count and percentage of various cells from the differential count, individual cell count for each immune cell type was calculated. BAL supernatant was used for cytokine analysis. IL-6 levels measured using the Luminex platform, specifically, the Mouse Custom ProcartaPlex 3-plex cytokine kit (Thermo Fisher Scientific, PPX-03-MXH6AC2) on the MAGPIX instrument.

### Biochemical Assays

Mouse tissues were homogenized in RIPA lysis buffer and the extract spun at 13200 g, 10 min, 4° C. Total cell lysates were electrophoresed and transferred to nitrocellulose or PVDF membrane and blocked in 5% milk in PBS containing 0.05% Tween-20. Membranes were incubated with primary antibodies overnight at 4° C. After incubation with HRP-conjugated secondary antibodies for 1 hr at room temperature, proteins were detected by chemiluminescence using SuperSignal West Pico Chemiluminescence or Luminata Forte Western HRP Substrate and Amersham Hyperfilms. The primary antibodies used: mouse monoclonal anti-mouse Sun1 clones X12.11 and X15.15, mouse anti-beta-actin (Sigma Aldrich, A1978). For hydroxyproline analysis, snap frozen lung samples were ground into powder using a mortar and pestle cooled with liquid nitrogen. ˜100 mg lung samples were analyzed for hydroxyproline content using the QuickZyme Hydroxyproline Assay kit (QuickZyme Biosciences).

### Statistical Analyses

Sample sizes (n) and test methods are specified in the figure legends for all the analyzed data. Significant differences between samples were accepted when P<0.05. Matlab and Origin version 2021b were used to test the significance and plots. Error bars in the scatter and bar graphs represent standard deviation of the mean. In box-whisker plots, whiskers extend from the minimum to the maximum value of the data set and the box represents the 25^{th} percentile to 75^{th} percentile of the data. Outliers are represented as a closed red circle and the line in the box represents the median of the data set.

### Results

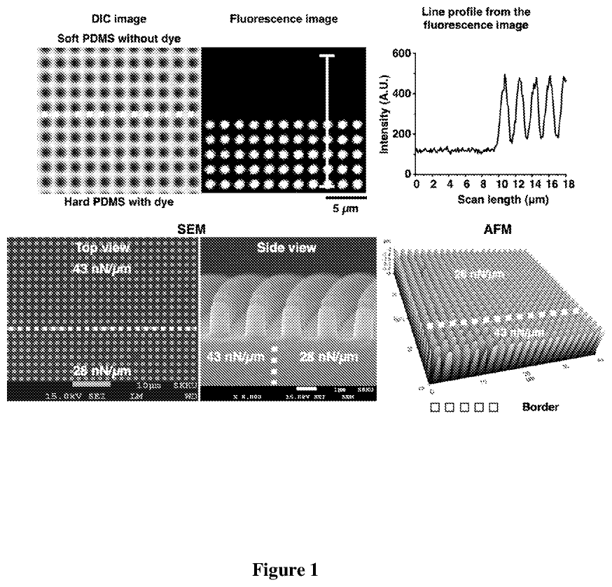

Durotaxis, or migration of cells from a soft to a stiff substrate, has been associated with various pathological processes, such as cancer metastasis and fibrosis. In order to study molecular regulators of durotaxis, a stepped stiffness gradient pillar array for measuring forces with subcellular resolution during cell migration was used (FIG. 1). The pillar array has two distinct stiffness—cells exhibiting durotaxis would preferentially migrate from the soft to hard substrate. Pillar deflection as the cell migrates enables biophysical quantification of forces at the subcellular level.

Molecular regulators of durotaxis are not well characterized. It was hypothesized that the LINC complex might be involved in durotaxis in some way. The LINC complex is thought to regulate cell migration through its role in nuclear positioning, but to date, its role in durotaxis has not been investigated. In order to do so, an immortalized fibroblast cell line was transduced with a lentivirus construct expressing a HA-tagged dominant negative Sun1 (HA-DNSun1) under the control of a doxycycline-inducible promoter.

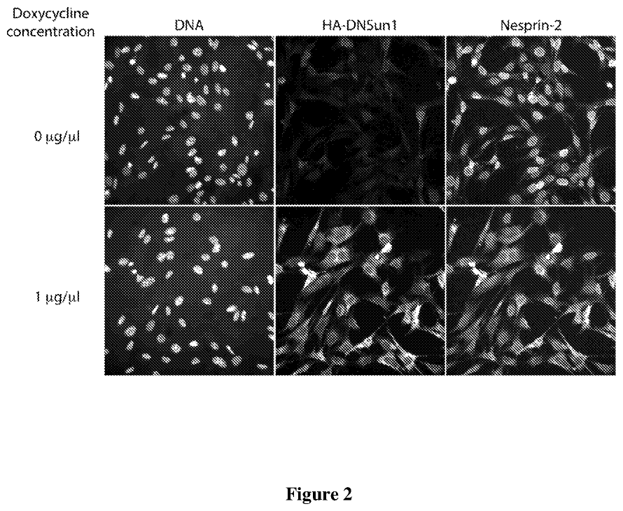

Expression of DNSun1 upon addition of doxycycline to cell culture media results in displacement of Nesprin-2 from the nuclear envelope of fibroblasts, indicating disruption of the LINC complex (FIG. 2).

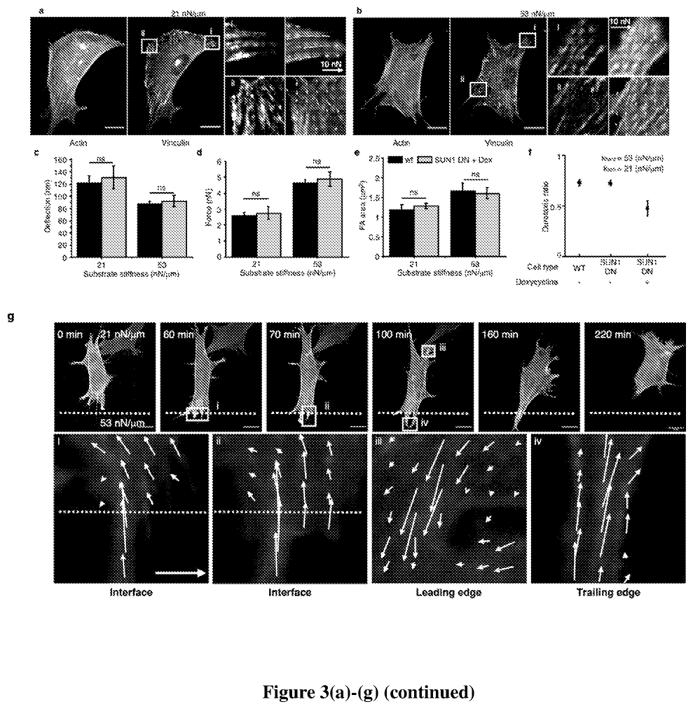

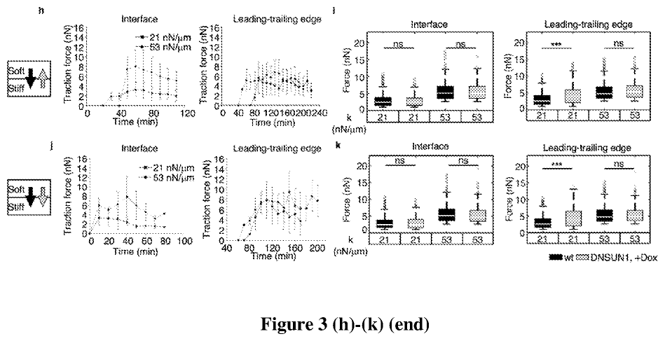

First, the behavior of wildtype fibroblasts and the fibroblasts harboring the DNSUN1 transgene were examined on pillar arrays with uniform substrate stiffnesses of 21 nN/mm or 53 nN/mm. In the presence of doxycycline and disrupted LINC complexes, cells showed the same response to the substrate rigidity compared to the wildtype fibroblasts or transgenic fibroblasts not treated with doxycycline (FIG. 3a-e). Next, low-magnification imaging was conducted on pillar arrays with two substrate stiffnesses (21 and 53 nN/m) to determine whether DNSUN1 expression affects durotaxis. Durotaxis is measured using a durotactic index, which is calculated by dividing the number of cells undergoing durotaxis by the total number of cells that touch the stiffness border. Thus, if the ratio is close to 1, it represents cells undergoing durotaxis. However, if the ratio is close to 0.5, it represents cells randomly migrating around the stiffness border. In the absence of the doxycycline, when the LINC complex is not disrupted, mutant cells showed durotaxis, as did the wild-type MEFs (FIG. 3f), with a durotactic index around 0.7. However, when the LINC complex is disrupted by the presence of doxycycline inducing DNSUN1 expression, mutant cells did not undergo durotaxis (FIG. 3f), exhibiting a durotactic index of 0.5.

Higher resolution imaging was then carried out to measure traction forces of migrating cells on soft compared to stiff substrates, through deflection of the nanopillars on the membrane (FIG. 3g-k). When a DNSun1-expressing cell migrates towards the 21 nN/m substrate after touching the 53 nN/μm substrate, the cell showed stiffness-dependent traction force at the interface area—with higher traction force at the 53 nN/μm substrate, and lower traction force at the 21 nN/μm substrate (FIG. 3g, h). However, DNSun1-expressing cells migrating from stiff to soft substrates exhibited increased traction force at the 21 nN/m substrate in the leading-trailing edge compared to at the interface (FIG. 3g, h). The regions of DNSun1-expressing cells at the interface, and the leading or trailing edges of these cells on the 53 nN/μm substrate as they migrate toward the 21 nN/μm substrate, generated similar traction forces to wild-type cells (FIG. 3i). In contrast, the leading or trailing edges of DNSun1-expressing cells on the 21 nN/μm substrate have increased traction force compared to wildtype cells (FIG. 3i). Strikingly, DNSun1-expressing cells migrating toward the 53 nN/m area showed similar results (FIG. 3j, k), where the leading or trailing edge on the 21 nN/μm substrate exhibited increased traction forces compared to wildtype cells.

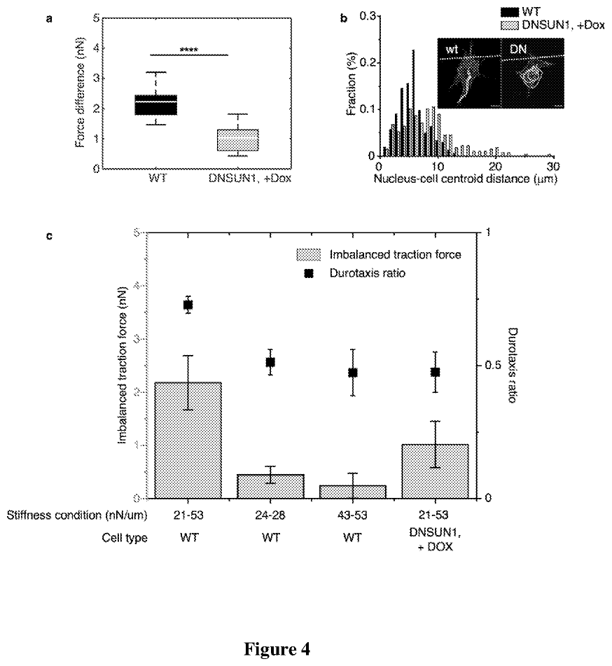

The data suggests that durotaxis occurs because of the imbalance of traction forces in a cell exploring two stiffness regimes—high traction force on the stiff substrate, and low traction force on the soft substrate. This imbalance of forces promotes cell migration towards the stiffer substrate. In DNSun1-expressing cells, this imbalance of traction forces is disrupted, with the cells generating comparably high traction forces on the stiff and soft substrates, resulting in low force difference or low force imbalance (FIG. 4a). A possible explanation is coupling of the nucleus to the actin cytoskeleton by the LINC complex usually compensates for the force imbalance generated by the cell being on two stiffness regimes. In the absence of nuclear-cytoskeleton coupling by the LINC complex upon DNSun1 expression, the actin cytoskeleton generates additional forces in the region of the migrating cell on the soft substrate to compensate for the force imbalance. Consequently, DNSun1-expressing cells have a larger cell centroid to nuclear centroid distance than wildtype cells, and appear to be unable to migrate directionally (FIG. 4b). While the exact mechanism remains to be determined, it is apparent that LINC complex disruption prevents the cell from distinguishing soft and stiff substrates through imbalances in local traction force generation, thereby inhibiting durotaxis (FIG. 4c).

As such, it has been shown that LINC complex disruption through exogenous delivery of DNSun1 inhibits durotaxis.

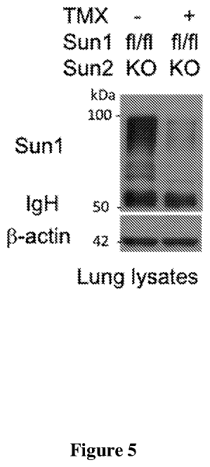

Since modulating durotaxis has been thought to be a potential therapeutic target for fibrosis, it was decided to validate the LINC complex as a therapeutic target for fibrosis. A mouse model where LINC complexes could be conditionally ablated was established. LINC complexes are comprised of SUN domain proteins of the inner nuclear membrane that physically interact with KASH domain proteins of the outer nuclear membrane. Two SUN domain proteins, Sun1 and Sun2, are broadly expressed in virtually all tissues—it appears that all LINC complexes are comprised of Sun1 or Sun2. Constitutive Sun1/2 double mutant mice suffer perinatal lethality. To study LINC complex function in adults, Sun1 “knockout first, conditional ready” mice (Sun1^{tm1a}) was obtained from the International Mouse Phenotyping Consortium, which were crossed to β-actin-Flp mice to obtain conditional knockout Sun1^{tm1c} mice. To ablate the LINC complex in vivo, a mouse line harbouring conditional alleles for Sun1 (Sun1^{tm1c/tm1c}) and null alleles for Sun2 (Sun2^{-/-}), and a transgene for tamoxifen-regulated Cre recombinase at the safe harbour Rosa26 locus (Rosa26^{MerCreMer/MerCreMer}) was established. Following intraperitoneal injection of tamoxifen in 6-week-old mice, lungs were harvested at 8 weeks and Western blots for Sun1 were performed (FIG. 5). In mice treated with vehicle (corn oil), Sun1 was well-expressed in the lungs. In mice treated with tamoxifen, Sun1 was almost completely depleted in lung tissue. Thus Sun1^{tm1c/tm1c};Sun2^{-/-};Rosa26^{MerCreMer} mice treated with tamoxifen (TMX) are effectively Sun1/2 double mutant in the lung.

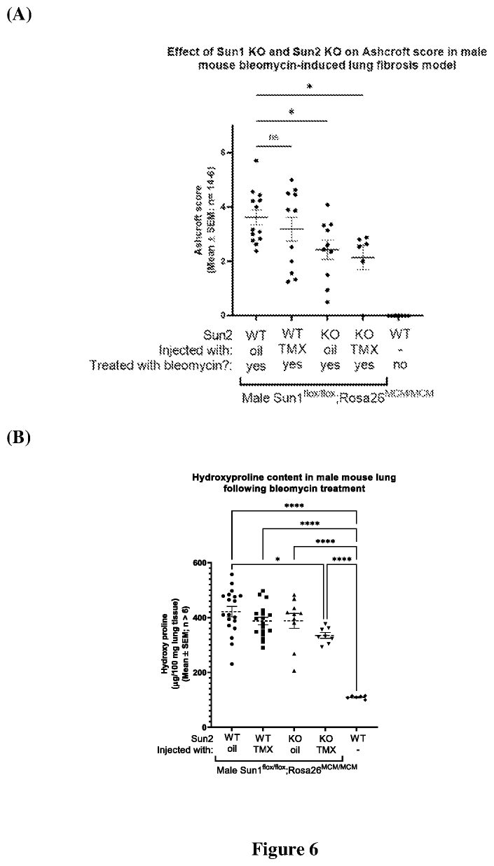

To investigate the role of the LINC complex in fibrosis in vivo, male mice were exposed to bleomycin intratracheally in a standard lung fibrosis model. Mice that were effectively wildtype (Sun1^{tm1c/tm1c};Sun2^{+/+};Rosa26^{MerCreMer} vehicle treated), Sun1 single “knockout” (Sun1^{tm1c/tm1c};Sun2^{+/+};Rosa26^{MerCreMer} tamoxifen treated), Sun2 single “knockout” (Sun1^{tm1c/tm1c};Sun2^{-/-};Rosa26^{MerCreMer} vehicle treated) and Sun1/Sun2 double “knockout” (Sun1^{tm1c/tm1c};Sun2^{-/-};Rosa26^{MerCreMer} tamoxifen treated) were used and sacrificed three weeks later. Histopathological evaluation of lung tissue was carried out to assign Ashcroft scores for the purpose of quantifying the extent of fibrosis. Lung tissue from Sun2 knockout and Sun1/Sun2 double knockout animals had reduced Ashcroft scores compared to wildtype animals (FIG. 6A). A biomarker of lung fibrosis is lung hydroxyproline content, a biochemical measure of collagen in the lungs, a component of fibrotic extracellular matrix. Loss of both Sun1 and Sun2 reduced lung hydroxyproline content by more than 25% (FIG. 6B). Thus disruption of LINC complexes through loss of Sun2 or a combination of Sun1 and Sun2 can suppress lung fibrosis.

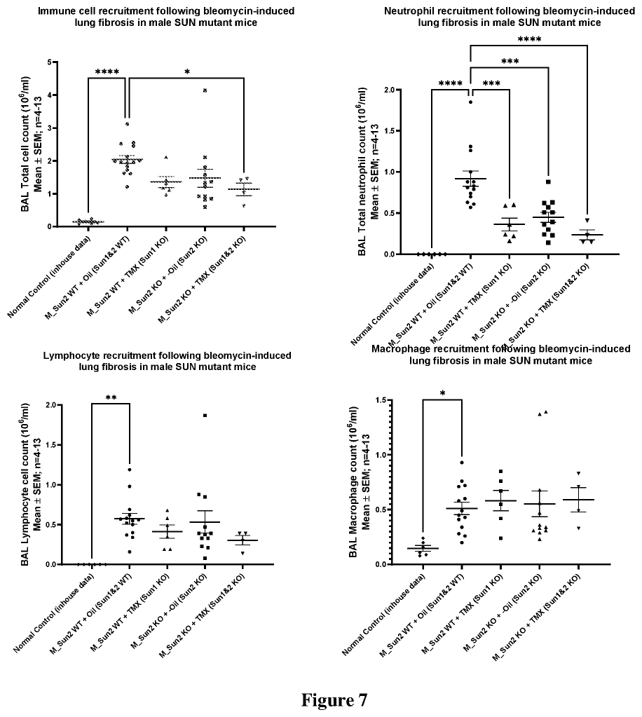

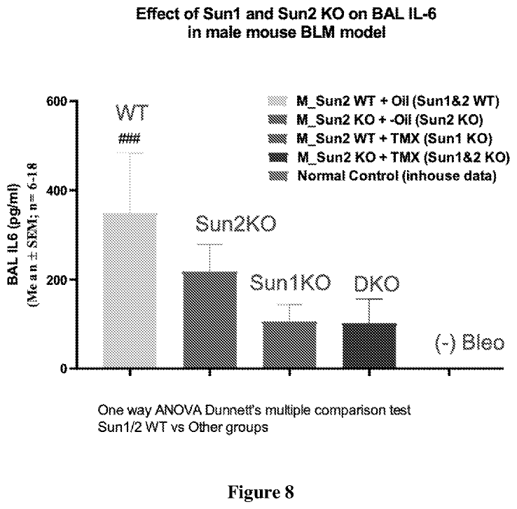

To further understand biological processes associated with lung fibrosis, BAL (bronchoalveolar lavage) fluid collected from lung via tracheal cannulation were analysed for inflammatory cells. Bleomycin treated mice exhibited elevated levels of immune cells in BAL fluid compared to untreated mice. Interestingly, Sun1/2 double mutant mice had almost half the amount of immune cells present in BAL fluid compared to wildtype controls (FIG. 7). While lymphocyte and macrophage recruitment were largely similar across the 4 genetic backgrounds, neutrophil counts were very much reduced following disruption of either Sun1 or Sun2, or both Sun1 and Sun2. BAL supernatants were also analyzed for the inflammatory cytokine IL-6, which was reduced in samples obtained from Sun1 mutant, Sun2 mutant and Sun1/2 double mutant mice (FIG. 8).

The reduction in neutrophil recruitment and IL-6 levels following bleomycin injury in mice suggests that LINC complex disruption via mutations in Sun1 and/or Sun2 reduces lung inflammation and reflects attenuation of the bleomycin-induced fibrotic response, showing that LINC complex disruption ameliorates pulmonary fibrosis in vivo.

### Example 2

### Methods

### Mouse Experiments

All mice were maintained under protocols approved by the Institutional Animal Care and Use Committee (IACUC). Male Sun2^{+/+};Sun1^{flox/flox} and Sun2^{-/-};Sun1^{flex/flox} mice were weaned at 3 weeks of age. At 6 weeks of age, tamoxifen was administered to mice via intraperitoneal injection, at a dose of 50 mg/kg, every 24 hours, for 3 consecutive days to drive global deletion of Sun1 in mice. In control experiments with the Sun1 gene intact, mice were injected following the same regime but with corn oil. At 8 weeks of age, the mice were then introduced to a Methionine and Choline deficient diet (Research Diets Inc, A0208202BRi) or regular chow for 8 weeks. Blood serum and livers were then isolated for downstream analysis following euthanasia via CO₂ intoxication. Liver histology, including Masson's trichrome, Picrosirius Red, and Oil Red O staining, were performed using standard methods at the A*STAR Advanced Molecular Pathology Laboratory (AMPL).

### Molecular Biology

To generate lentiviral constructs expressing shRNA, appropriate oligos were ordered, annealed, and cloned into pLKO.1-blast (Addgene #26655). The shRNA sequences used are shown in the table below in sense-hairpin-antisense format.

| | |
|---|---|
| scrambled shrna | TCAACAAGATGAAGAGCACCGA CTCGAG |
| | TTGGTGCTCTGCATCTTGTTG |
| | (SEQ ID NO: 98) |
| Mouse Sun1 | AGGCTATTGATTCGCACATTA ctcgag |
| | TAATGTGCGAATCAATAGCCT |
| | (SEQ ID NO: 99) |
| Mouse Sun2 | GCACTGTTGATTCTTTCAA TTGGATCCAA |
| | TTGAAAGAATCAACAGTGC |
| | (SEQ ID NO: 100) |
| Human Sun1 | GCTGTTCTGAAACTTACGAAACTCGAGTTTCGT |
| | AAGTTTCAGAACAGC |
| | (SEQ ID NO: 101) |
| Human Sun2 | GCCTATTCAGACGTTTCACTTCTCGAGAAGTGA |
| | AACGTCTGAATAGGC |
| | (SEQ ID NO: 102) |

A construct encoding dominant negative SUN1 with an EGFP tag was generated using fusion PCR, and cloned into a modified pTripZ (Dharmacon) lentiviral plasmid. The EGFP-DNSUN1 sequence comprises the first 25 amino acids of human serum albumin encompassing the signal peptide, EGFP, the last 453 amino acids of human SUN1, and the ER-Golgi retrieval signal KDEL, with the EGFP flanked by polylinkers.

| | |
|---|---|
| EGFP-DNSUN1 | MKWVTFISLLFLFSSAYSRGVFRRDGPSGGVSKG |
| | EELFTGVVPILVELDGDVNGHKFSVSGEGEGDAT |
| | YGKLTLKFICTTGKLPVPWPTLVTTLTYGVQCFSR |
| | YPDHMKQHDFFKSAMPEGYVQERTIFFKDDGNY |
| | KTRAEVKFEGDTLVNRIELKGIDFKEDGNILGHKL |
| | EYNYNSHNVYIMADKQKNGIKVNFKIRHNIEDGS |
| | VQLADHYQQNTPIGDGPVLLPDNHYLSTQSALSK |
| | DPNEKRDHMVLLEFVTAAGITLGMDELYKTGGD |
| | DPQDVFKPTTSRLKQPLQGDSEAFPWHWMSGVE |
| | QQVASLSGQCHHHGENLRELTTLLQKLQARVDQ |
| | MEGGAAGPSASVRDAVGQPPRETDFMAFHQEHE |
| | VRMSHLEDILGKLREKSEAIQKELEQTKQKTISAV |
| | GEQLLPTVEHLQLELDQLKSELSSWRHVKTGCET |
| | VDAVQERVDVQVREMVKLLFSEDQQGGSLEQLL |
| | QRFSSQFVSKGDLQTMLRDLQLQILRNVTHHVSV |
| | TKQLPTSEAVVSAVSEAGASGITEAQARAIVNSAL |
| | KLYSQDKTGMVDFALESGGGSILSTRCSETYETK |
| | TALMSLFGIPLWYFSQSPRVVIQPDIYPGNCWAFK |
| | GSQGYLVVRLSMMIHPAAFTLEHIPKTLSPTGNIS |
| | SAPKDFAVYGLENEYQEEGQLLGQFTYDQDGESL |
| | QMFQALKRPDDTAFQIVELRIFSNWGHPEYTCLY |
| | RFRVHGEPVKKDEL (SEQ ID NO: 103) |
| Signal peptide derived | MKWVTFISLLFLFSSAYSRGVFRRD (SEQ ID NO: 104) |
| from human serum | |
| albumin | |
| EGFP (enhanced green | VSKGEELFTGVVPILVELDGDVNGHKFSVSGEGE |
| fluorescent protein) tag | GDATYGKLTLKFICTTGKLPVPWPTLVTTLTYGV |
| | QCFSRYPDHMKQHDFFKSAMPEGYVQERTIFFKD |
| | DGNYKTRAEVKFEGDTLVNRIELKGIDFKEDGNIL |
| | GHKLEYNYNSHNVYIMADKQKNGIKVNFKIRHNI |
| | EDGSVQLADHYQQNTPIGDGPVLLPDNHYLSTQS |
| | ALSKDPNEKRDHMVLLEFVTAAGITLGMDELYK |
| | (SEQ ID NO: 105) |
| Human Sun1 360-812 | DDPQDVFKPTTSRLKQPLQGDSEAFPWHWMSGV |
| amino acids from | EQQVASLSGQCHHHGENLRELTTLLQKLQARVD |
| NP_001354617 | QMEGGAAGPSASVRDAVGQPPRETDFMAFHQEH |
| | EVRMSHLEDILGKLREKSEAIQKELEQTKQKTISA |
| | VGEQLLPTVEHLQLELDQLKSELSSWRHVKTGCE |
| | TVDAVQERVDVQVREMVKLLFSEDQQGGSLEQL |
| | LQRFSSQFVSKGDLQTMLRDLQLQILRNVTHHVS |
| | VTKQLPTSEAVVSAVSEAGASGITEAQARAIVNSA |
| | LKLYSQDKTGMVDFALESGGGSILSTRCSETYET |
| | KTALMSLFGIPLWYFSQSPRVVIQPDIYPGNCWAF |
| | KGSQGYLVVRLSMMIHPAAFTLEHIPKTLSPTGNI |
| | SSAPKDFAVYGLENEYQEEGQLLGQFTYDQDGES |
| | LQMFQALKRPDDTAFQIVELRIFSNWGHPEYTCL |
| | YRFRVHGEPVK (SEQ ID NO: 106) |
| ER-to-Golgi retrieval | KDEL (SEQ ID NO: 107) |
| signal | |

Transient siRNA Depletion in Mice

In vivo siRNA was performed to deplete Sun2 from the liver of C57BL/6NTac male mice (age 7-8 weeks, weighing approx. 20-25 g) using ThermnoFisher Invivofectamine 3.0.

The siRNA duplex solution and the final injection solution were prepared according to the manufacturer's protocol. To begin with, siRNA (negative control siRNA. #4404020; Sun2 siRNA #4457′108) was mixed with an equal volume of complexation buffer. The solution was then mixed with an equivalent volume off Invivofectamine 3.0 reagent (ThermoFisher #IVF3005) and brought to vortex immediately. The siRNA duplex mixture was incubated at 50° C. for 30 min, then diluted 6-fold in PBS. The Sun-2 siRNA (s104591) was an Ambion pre-designed sequence: sense CUCUCAGGAUGAUAACGAUTT (SEQ ID NO: 108); antisense (5′-3′): AUCGUUAUCAUCCUGAGAGTA (SEQ ID NO: 109). The solution had a final concentration at 0.1 mg/mL. The solution was injected by the retro-orbital route into mice at a dosage of 10 uL/g. In pilot experiments, mice were injected once with siRNA, and harvested 5 and 7 days later. For mice treated with methionine-choline deficient diet, injections were performed every 7 days for a period of 8 weeks before mice were sacrificed for analysis.

### Western Blot

Liver tissue was harvested after mice were sacrificed. The tissue was transferred to a tube with 1% SDS buffer (1% (w/v) SDS, 10 mM TRIS, 1 mM EDTA, pH 8.0) at a ratio of 37.5 mg tissue to 1 ml buffer. Tubes containing tissue and buffer were transferred to a homogenizer for homogenization. Tissue lysate was separated from cell debris by centrifugation at 10,000 g for 20 min. Tissue lysate was collected and samples were sonicated on a Fisher Scientific Model 505 Sonic Dismembrator for 15 s and 2 cycles to shear DNA. Total protein concentration was determined using a bicinchoninic acid (BCA) protein assay kit (Pierce, Catalogue number: 23225). Equal protein amounts were mixed with 4× Laemmli sample buffer, heated for 5 min at 95° C., loaded on 8% SDS-PAGE gels and transferred onto a nitrocellulose membrane through wet transfer. Membranes were blocked in 5% milk/0.1% Tween-PBS and incubated with primary antibodies from 1 hour to overnight at 4° C. After incubation with HRP-conjugated secondary antibodies for 45 min at room temperature, proteins were detected by chemiluminescence using SuperSignal West Pico Chemiluminescence (Thermo Fisher Scientific, Cat #34080), or SuperSignal West Femto Maximum Sensitivity Substrate (Thermo Fisher Scientific, Cat #34095) with an Amersham ImageQuant 800 imager.

### Hepatic Stellate Cell (HSC) Isolation

Hepatic stellate cells were isolated using a modified protocol that was previously published. Briefly, mice were euthanized using CO₂ intoxication. Each mouse liver was perfused with 10 mL of ice cold 1×HBSS via the hepatic portal vein. The inferior vena cava was immediately cut to release the pressure. The livers were then dissected out, the gallbladders removed and washed with 1×HBSS. Livers from 4 mice were then pooled, minced and digested using 20 mL of a prewarmed cocktail consisting of 0.1% Collagenase B and 0.5% Pronase in 1×HBSS. 2 ml of 0.025% DNAseI was then supplemented to prevent cell clumping. The mixture was mixed and placed in an incubator-shaker at 37° C. for 20 min. The digested mixture was then strained through a 100 μm cell strainer and the flow through was collected. The flow through was centrifuged at 50×g for 1 min, the supernatant was then centrifuged again at 50×g for 1 min to remove most of the parenchymal cells. The supernatant was then centrifuged at 300 g to pellet the non-parenchymal cells. The non-parenchymal cells were further purified with a Percoll gradient. Specifically, the cells were resuspended in 9.3 ml of DMEM and mixed with 12.7 ml of Percoll containing 10% volume of 10×PBS. After centrifugation at 20,000×g for 10 minutes at 4 deg, the cell layer above the 1.07 gradient was carefully recovered. The recovered cells were then stained with FITC conjugated CD11b followed by FACs sorting. Purified HSCs were obtained from the population of CD11b negative cells. HSCs were subsequently maintained in high glucose DMEM supplemented with 20% FBS, 1% Penicillin-Streptomycin and 1% L-glutamine

### Cell Culture

Primary human dermal fibroblasts from foreskin (21-S-ASB-010 FIBRO) were obtained from the Asian Skin Biobank. Isolated HSCs, dermal fibroblasts, and IMR90 human fetal lung fibroblasts were cultured at 37° C. in high glucose DMEM supplemented with 20% FBS, 1% Penicillin-Streptomycin and 1% L-glutamine.

For TGFβ activation studies, cells were first serum starved by culturing in high glucose DMEM supplemented with 2% horse serum and 1% Penicillin-Streptomycin for 24 hours. TGFβ was then added at 10 ng/mL for 48 hours in serum starvation media. In parallel, control non-TGFβ treated cells were maintained in serum starvation media Cells were then harvested for western analysis.

### Albumin, ALT and AST Biochemical Assays

Biochemical assays for quantifying serum albumin (Elabscience E-BC-K057-M), ALT (Elabscience E-BC-K235-M) and AST (Elabscience E-BC-K236M) levels were carried out following manufacturer instructions.

### Picrosirius Red Analysis

Picrosirius red analysis was done using an in-house written ImageJ macro. Images were first converted to 8-bit. To obtain the area fraction of the liver stained by Picrosirius red (PSR), two default thresholding algorithms were applied to each image separately. Specifically, the MaxEntropy threshold gives the area that is stained by PSR and the Mean threshold yields the area of the entire liver section imaged. The ratio of the resulting two areas yields the fraction of the liver sections stained by PSR (normalized fibrosis scores)

### Results

In order to further validate the LINC complex as a therapeutic target for fibrosis, a mouse model where LINC complexes could be conditionally ablated was established. LINC complexes are comprised of SUN domain proteins of the inner nuclear membrane that physically interact with KASH domain proteins of the outer nuclear membrane. Two SUN domain proteins, Sun1 and Sun2, are broadly expressed in virtually all tissues—it appears that all LINC complexes are comprised of Sun1 or Sun2. To study LINC complex function in adults, Sun1 “knockout first, conditional ready” mice (Sun1^{tmla}) were obtained from the International Mouse Phenotyping Consortium, and were crossed to b-actin-Flp mice to obtain conditional knockout Sun1^{tm1c} mice. To ablate the LINC complex in vivo, a mouse line harbouring conditional alleles for Sun1 (Sun1^{lm1c/tm1c}) and null alleles for Sun2 (Sun2^{-/-}), and a transgene for tamoxifen-regulated Cre recombinase at the safe harbour Rosa26 locus (Rosa26^{MerCreMer/MerCreMer}) was established. To delete Sun1, intraperitoneal injection of tamoxifen was carried out.

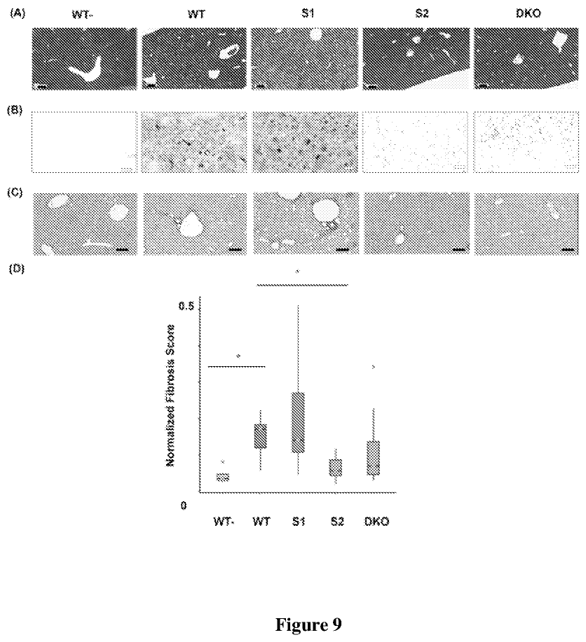

To investigate the role of the LINC complex in NASH and liver fibrosis in vivo, mice were put on a methionine-choline deficient (MCD) diet, with wildtype mice on normal chow as no diet controls. Mice that were effectively wildtype (Sun1^{tm1c/tm1c};Sun2^{2+/+};Rosa26^{MerCreMer} oil vehicle treated), Sun1 single “knockout” (Sun1 KO, Sun1^{tm1c/tm1c};Sun2^{+/+};Rosa26^{MerCreMer} tamoxifen treated), Sun2 single “knockout” (Sun2 KO, Sun1^{tm1c/tm1c};Sun2^{-/-};Rosa26^{MerCreMer} oil vehicle treated) and Sun1/Sun2 double “knockout” (Sun1/2 DKO, Sun1^{tm1c/tm1c};Sun2^{-/-};Rosa26^{MerCreMer}, tamoxifen treated) were used and sacrificed 8 weeks after being placed on the MCD diet. Histological assessment using Masson's trichrome and Oil Red O stains revealed liver steatosis in Sun1/2 wildtype and Sun1 KO mice on the MCD diet, which was considerably reduced in Sun2 KO and Sun1/2 DKO mice (FIG. 9A, B). Picrosirius red staining was also performed to evaluate liver fibrosis (FIG. 9C). Quantification of fibrotic areas showed increase in fibrosis in wildtype and Sun1KO mice, which was reduced in Sun2KO and Sun1/2DKO mice. Thus loss of LINC complexes can suppress NASH-associated liver steatosis and fibrosis.

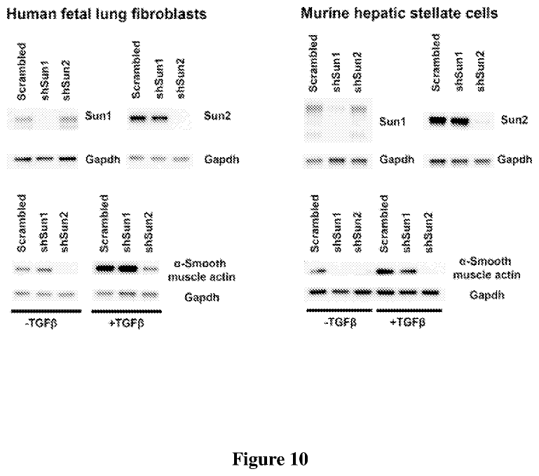

TGFβ is regarded as the master regulator of fibrosis. To understand potential mechanisms underlying the role of the LINC complex in fibrosis, TGFβ signaling responses in lung fibroblasts and hepatic stellate cells, specifically expression of the fibrosis marker, smooth muscle actin were investigated. The cell types investigated were significant, as they are thought to differentiate into fibrosis-driving myofibroblasts upon TGFb treatment. Sun1 and Sun2 were depleted by lentiviral delivery of shRNA in both cell types (FIG. 10, top panels). Upon treatment with TGFb, expression of smooth muscle actin (SMA) was increased in control cells and Sun1-depleted cells (FIG. 10, bottom panels). However, SMA levels did not increase in Sun2 depleted cells following TGFb treatment (FIG. 10, bottom panels). Here, it was shown that application of Sun2 RNAi was able to attenuate fibrosis in lung fibroblasts and hepatic stellate cells, which are regarded as the progenitor cells for myofibroblasts in the lung and liver. Thus Sun2 RNAi may be used for treatment of for lung and liver fibrosis.

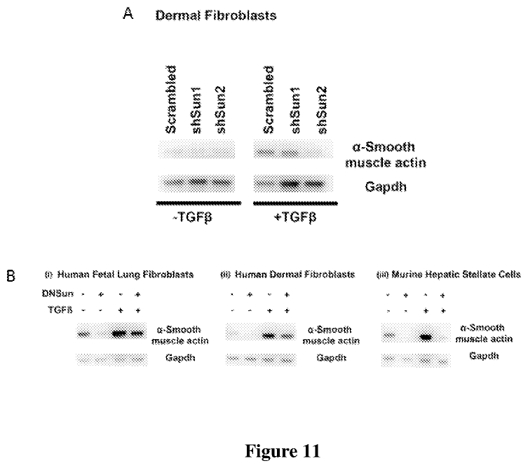

To investigate the role of the LINC complex in an additional form of fibrosis, skin fibrosis, dermal fibroblasts were depleted of Sun1 and Sun2 as described above. Upon treatment with TGFb, expression of smooth muscle actin (SMA) was increased in control cells and Sun1-depleted cells (FIG. 11A). However, SMA levels did not increase in Sun2 depleted cells following TGFb treatment (FIG. 11A). Here, it was shown that application of Sun2 RNAi was able to attenuate fibrosis in dermal fibroblasts, which are regarded as the progenitor cells for myofibroblasts in the skin. Thus Sun2 RNAi may be used for treatment of for skin fibrosis.

To investigate an alternate approach to LINC complex disruption for the treatment of fibrosis, lung fibroblasts, dermal fibroblasts and hepatic stellate cells were transduced with a lentiviral construct harbouring a doxycycline-inducible dominant negative Sun1 (DNSUN1) transgene. The DNSUN1 transgene abolishes SUN-KASH protein-protein interactions by competitively binding to KASH domains of KASH domain proteins. Expression of DNSUN1 attenuates smooth muscle actin expression both at baseline and in response to TGFb treatment (FIG. 11B). Here, it is shown that disrupting the SUN-KASH protein-protein interaction by expression of DNSUN1 attenuates fibrosis in lung fibroblasts, dermal fibroblasts, and hepatic stellate cells. Thus expression of DNSUN1 or other inhibitor of SUN-KASH protein-protein interaction may be used for treatment of lung, skin or liver fibrosis

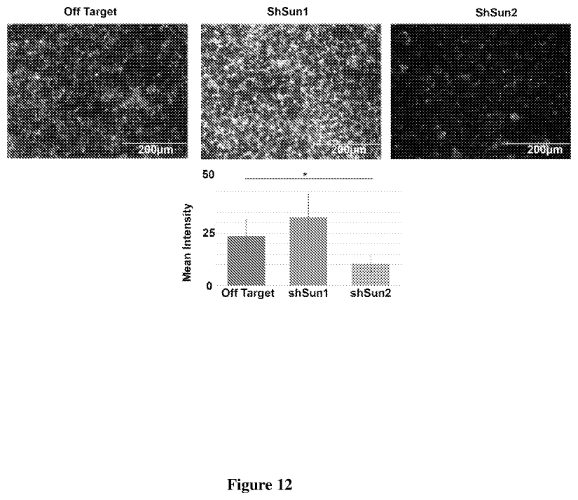

To further validate the role of the LINC complex in steatosis, Huh-7 hepatocyte cells were loaded with BODIPY FL C₁₆ green fluorescent fatty acid following depletion of Sun1 or Sun2 by shRNA. There was a statistically significant decrease in fluorescent staining of lipids in Sun2-depleted cells compared to control cells, indicating that Sun2 was required for lipid accumulation (FIG. 12). Here, it was shown that application of Sun2 RNAi was able to attenuate steatosis in hepatocytes. Thus Sun2 RNAi may be used for treatment of liver steatosis.

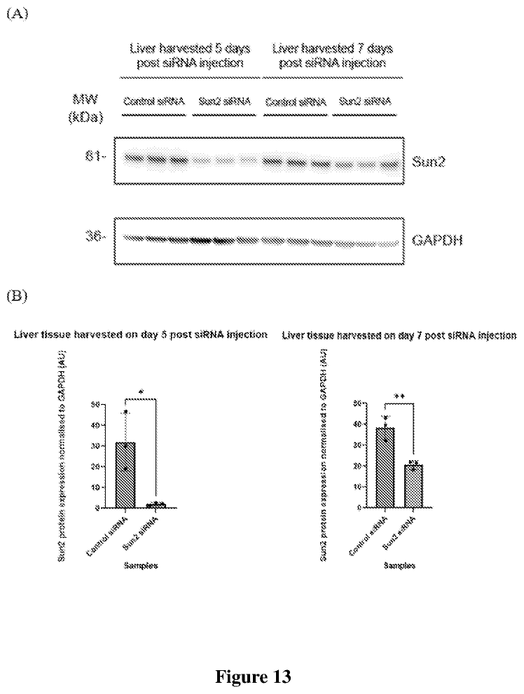

In order to target the LINC complex in vivo to potentially treat liver steatosis and fibrosis, a lipid nanoparticle, Invivofectamine, was used to transfect control or Sun2 siRNA in mice following intravenous delivery. A significant decline in Sun2 protein level in the liver was observed 5 to 7 days after transfection (FIG. 13). Thus it is possible to pharmacologically inhibit LINC complexes by depleting Sun2 using siRNA transfection in the liver.

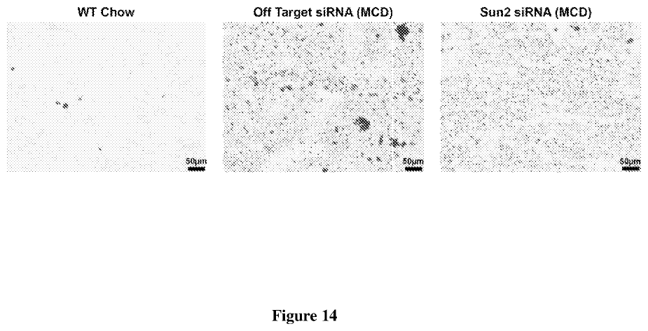

To further validate the role of Sun2 in liver steatosis in an animal model, mice were put on a control normal chow diet or on a methionine-choline deficient (MCD) diet that models non-alcoholic steatohepatitis (NASH). Mice on the MCD diet were injected weekly with control or Sun2 siRNA as described above. 8 weeks later, mice were sacrificed and their livers analyzed for steatosis by Oil Red O staining. Oil Red O staining indicated a reduction in lipid accumulation in Sun2 siRNA liver compared to control (Off Target) siRNA liver (FIG. 14). Here, it was shown that application of Sun2 RNAi was able to attenuate steatosis in the liver. Thus Sun2 RNAi may be used for treatment of liver steatosis.

### Example 3

### Methods

Both control C57BL6 animals and Sun1/Sun2 double “knockout” (Sun1/2 DKO Sun1tm1c/tm1c; Sun2−/−;Rosa26MerCreMer, tamoxifen treated) were used to investigate the role of the LINC complex in wound healing in vivo. Both males and females were used for experiments as no obvious differences were observed between sexes. Mice were housed individually, in Biological Resource Centre of A*STAR, at Immunos building. Animals were handled, bred and euthanized in compliance with the guidelines and procedures approved by the A*STAR IACUC (Institutional Animal Care and Use Committee). Animals were regularly monitored for any health concerns. All animals for experiments were housed in a specific pathogen free facility in ventilated cages kept under 12-hour light and dark cycle and were given unlimited food and water. The temperature in the facility was maintained at 21° C.

Briefly, 8 weeks old animals were used for performing the full thickness wound. Two bilateral full thickness wounds of 4 mm diameter each was created using sterile biopsy punch on both sides of anesthetized mouse's midline at the shoulder level. Wounds were regularly monitored and imaged to analyse the wound closure. Animals were sacrificed on day 6 to harvest skin tissue containing the wounds area for further analysis.

Skin sections from the back skin of the mice containing the wound were either fixed in 10% NBF and further processed to generate paraffin blocks or freshly embedded in OCT. Skin tissue sections were further analysed by histological assays like H&E. Masson's trichrome stain, and immunohistochemistry.

### Results

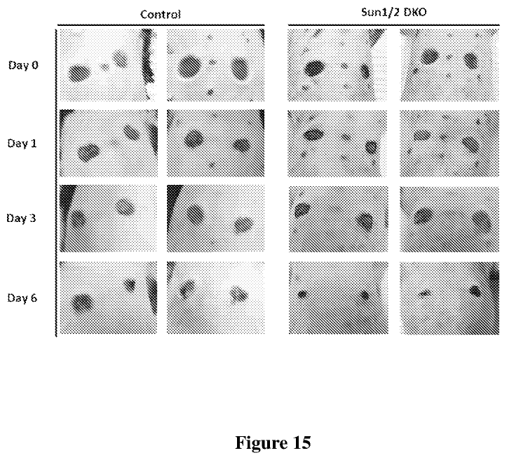

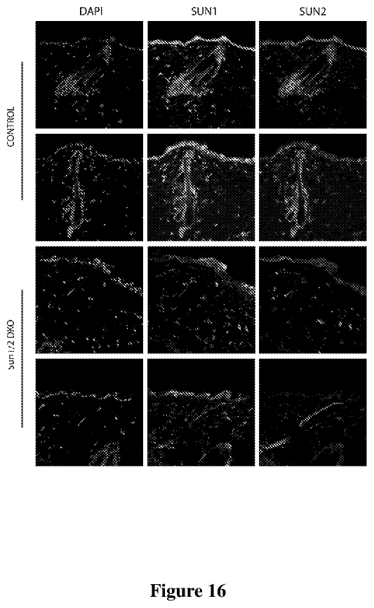

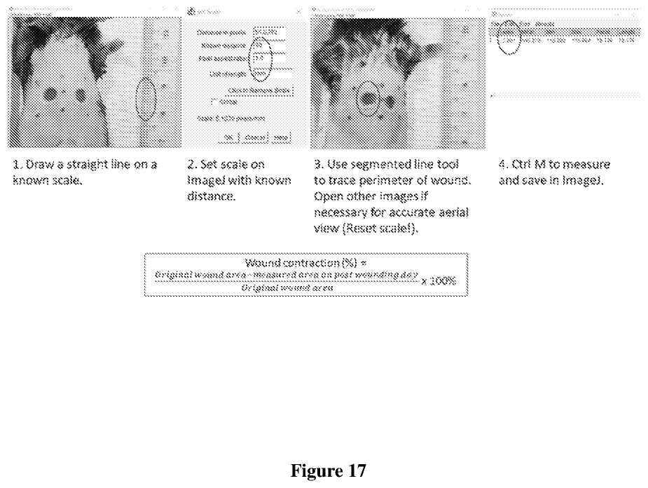

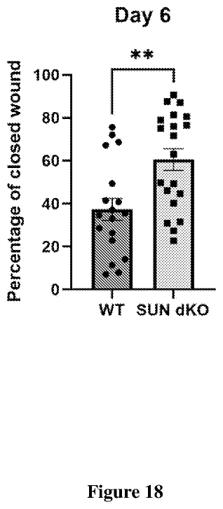

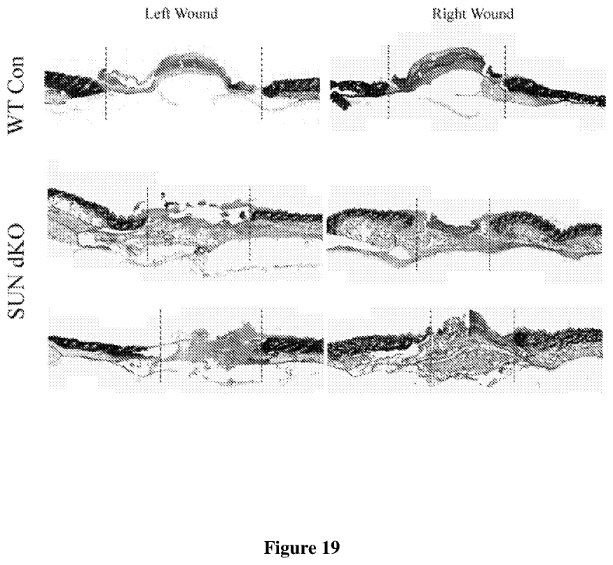

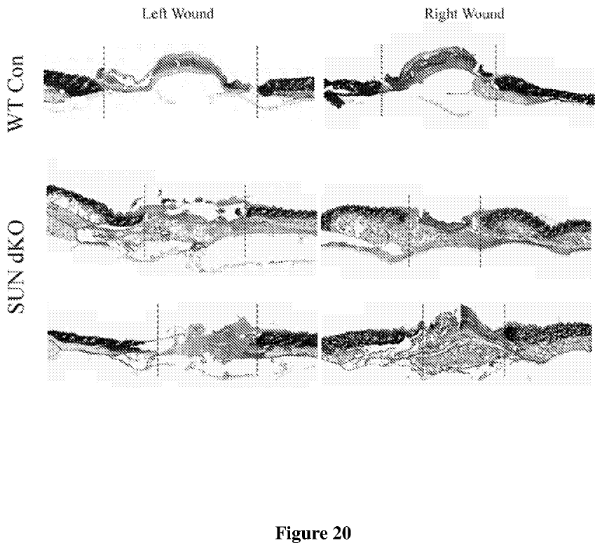

To explore the role of LINC complex in maintaining skin homeostasis, a full thickness wound of 4 mm diameter was performed on the back skin of control and SUN1/2 DKO mice. Digital images of the wound area were used to the wound area every alternate days starting from the day 0 of the wound to day 6 (FIG. 15). To confirm the absence of SUN domain proteins in the SUN1/2 DKO mice, biopsies collected upon wounding were sectioned and analysed by immunofluorescence (FIG. 16). Wound areas were measured by using a segmented line tool on image J to trace the area of the wound and calculated by the scale set. Wound contraction was calculated by taking the original wound area minus the area measured on post wounding day over the original wound area and taken as a percentage (FIG. 17). Absence of LINC complex in the SUN1/2 DKO mice resulted in significant accelerated closure of the wound on day 6 compared to the control animals (FIG. 18-20).

### Example 4

### Methods

### Mouse Genetics

Mouse strains (FVB/N and C57BL/6) were maintained at the A*STAR Biological Resource Centre facility on a 12 h light/dark cycle in ventilated animal barrier facilities with the temperature set to 21±1° C., humidity at 55-70% and with food and water provided ad libitum. Ethical oversight was provided and approval granted by the Institutional Animal Care and Use Committees, for A*STAR Biological Resource Centre (BRC). BRC is governed by AAALAC guidelines provided to both AALAS (USA) and AVS (Singapore) to which A*STAR adheres. PyMT Sun1^{-/-} (mixed C57BL/6 and FVB/N background) mice were generated by crossing PyMT mice (FVB/N-Tg(MMTV-PyVT)634Mul/J, JAX stock 002374) and Sun1^{+/-} (C57BL/6 background) mice. To derive mice with a global deletion of Sun1, mice harbouring floxed Sun1 alleles (Sun1^{Flox/Flox}, described above) were crossed to mice in which Cre recombinase is driven by the regulatory sequences of the mouse zone pellucida 3 gene (Zp3;Tg(Zp3-cre)93Knw, JAX stock 003651). The ZP3 promoter drives Cre expression in the female germline. Thus to generate recombined Sun1 null alleles, Sun1^{Flox/+}Zp3cre^{+/-} and Sun1^{Flox/Flox}Zp3cre^{+/-} females were crossed with wild type males to generate Sun1^{+/-} and Sun1^{-/-} mice. Sun1^{+/-} mice were used for breeding as Sun1^{-/-} mice have been shown to be reproductively defective (Chi et al., 2009).

### Tumour Volume Measurement

Mice were palpated twice weekly from 7 weeks of age to monitor mammary tumor development. Tumors were measured in two dimensions using calipers and tumor volume estimated using the standard calculation n for a sphere 4/3×3.14×a ×b² where a is the smaller diameter and b is the larger diameter.

### RT-PCR

For RT-PCR, total RNA was extracted from the superior lobe of the lung of 12 week-old mice using TRIZOL and Qiagen RNAeasy Kit. Complementary DNA (cDNA) was generated using the Applied Biosystems (ABI) High Capacity cDNA Reverse Transcription Kit. RT-PCR was performed using ABI PowerUp SYBR Green Master Mix according to manufacturer's instructions. PyMT primers were PyMT fwd (5′-ctgctactgcacccagacaa-3′ (SEQ ID NO: 93)) and PyMT rev (5′-gcaggtaagaggcattctgc-3′ (SEQ ID NO: 94)). RPLP0 was used as a reference gene, using primers RPLP0 fwd (5′-gatgcccagggaagacag-3′ (SEQ ID NO: 95)) and RPLP0 rev (5′-acaatgaagcattttggataatca-3′ (SEQ ID NO: 96)). Reactions were run according to the PowerUP SYBR Green Master Mix protocol with a 60° C. annealing temperature.

### Results

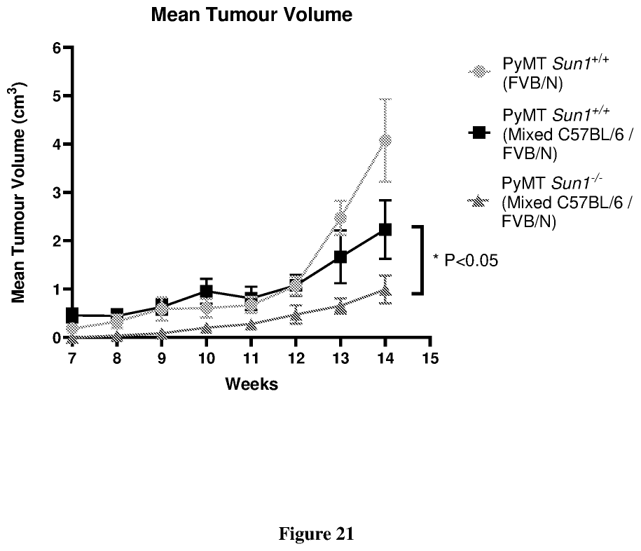

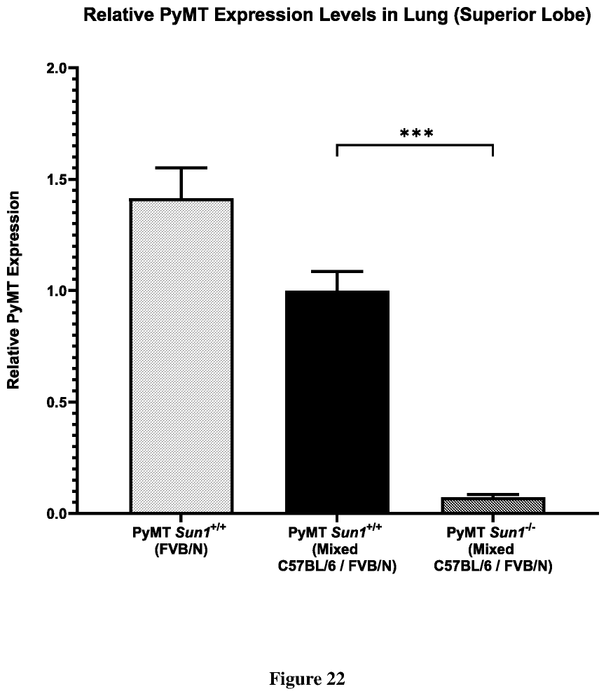

The MMTV-PyMT (mouse mammary tumor virus-polyoma middle T-antigen, PyMT) is a genetic model of metastatic breast cancer. Hemizygous PyMT females develop palpable mammary tumors which metastasize to the lung. To investigate the role of the LINC complex in tumour formation and metastasis, PyMT mice were crossed to Sun1 heterozygous null mice to obtain PyMT;Sun1^{+/+} or PyMT;Sun1^{-/-} mice. The genetic background of mice affects the development and metastasis of tumours in this model, with tumour formation and metastasis being promoted on the FVBN background and reduced on a C57BL/6 background. The Sun1 mice are on a C57BL/6 background. As such, while PyMT;Sun1^{+/+} mice on the original FVB/N background were monitored together with PyMT;Sun1^{+/+} mice on a mixed C57BL/6/FVB/N background, comparisons were only drawn between Sun1 wildtype and null PyMT mice on similar mixed C57BL/6/FVB/N backgrounds. The mice were monitored from 7 weeks of age onwards for the size of primary mammary tumours. Loss of Sun1 in the PyMT mixed background mice resulted in reduction in primary mammary tumour volume compared to PyMT mixed background mice wildtype for Sun1 (FIG. 21). To assess the level of metastasis, RT-PCR for the PyMT gene product, which is expressed only in cells of mammary origin due to the MMTV promoter, was carried out on RNA samples obtained from the superior lobe of the lung of 12 week-old mice. Loss of Sun1 results in a considerable reduction in PyMT expression in the lung, compared to the lungs of Sun1 wildtype PyMT mice (FIG. 22). These results indicate that loss of Sun1 significantly reduces the metastatic potential of MMTV-PyMT tumours.

1-26. (canceled)

27. A method of preventing or treating fibrosis or a disease associated with fibrosis in a subject, the method comprising administering an effective amount of a LINC complex inhibitor to the subject.

28. The method of claim 27, wherein the disease associated with fibrosis is skin, liver or lung fibrosis.

29. The method of claim 27, wherein the method comprises inhibiting durotaxis in the subject.

30. The method of claim 27, wherein the method comprises inhibiting TGFβ activity in the subject.

31. The method of claim 27, wherein the LINC complex inhibitor is a nucleic acid, polypeptide and/or small molecule.

32. The method of claim 27, wherein the LINC complex inhibitor is an inhibitor of a gene encoding SUN2, SUN1, Nesprin-1, Nesprin-2, Nesprin-3, Nesprin-4 or KASH5.

33. The method of claim 32, wherein the LINC complex inhibitor is an inhibitor of a gene encoding SUN2.

34. The method of claim 27, wherein the LINC complex inhibitor is an inhibitory nucleic acid molecule or site-specific nuclease (SSN) system that is capable of disrupting a gene encoding a LINC complex protein.

35. The method of claim 34, wherein the LINC complex inhibitor is an inhibitory nucleic acid molecule, wherein the inhibitory nucleic acid molecule is an siRNA, shRNA, ASO or miRNA.

36. The method of claim 34, wherein the LINC complex inhibitor is a site-specific nuclease (SSN) system, wherein the SSN system is a CRISPR-Cas system, optionally wherein the CRISPR-Cas system is a CRISPR-Cas9 system.

37. The method of claim 27, wherein the LINC complex inhibitor is a polypeptide derived from a LINC complex protein, optionally wherein the LINC complex inhibitor is a dominant negative SUN1 polypeptide.

38. The method of claim 37, wherein the LINC complex inhibitor is a polypeptide derived from SUN1 and/or SUN2 or is a polypeptide derived from Nesprin-1, Nesprin-2, Nesprin-3, Nesprin-4 and/or KASH5.

39. A method of inhibiting durotaxis of a cell, the method comprising contacting the cell with a LINC complex inhibitor.

40. The method of claim 39, wherein the cell is a hepatocyte, a myofibroblast cell, an immune cell or a cancer cell.

41. A method of treating a metastatic cancer in a subject, the method comprising administering an effective amount of a LINC complex inhibitor to the subject.
